(19) United States
(12) Patent Application Publication
(10) Pub. No.: US 2023/0256094 A1
(43) Pub. Date: Aug. 17, 2023

(54) CANCER RADIOSENSITIZATION BY IN SITU FORMATION OF GOLD NANOPARTICLES AND/OR GOLD NANOCLUSTERS

(71) Applicant: BOARD OF REGENTS, THE UNIVERSITY OF TEXAS SYSTEM, Austin, TX (US)

(72) Inventors: Aaron Star SCHWARTZ-DUVAL, Houston, TX (US); Sunil KRISHNAN, Ponte Vedra Beach, FL (US); Konstantin SOKOLOV, Missouri City, TX (US)

(73) Assignee: BOARD OF REGENTS, THE UNIVERSITY OF TEXAS SYSTEM, Austin, TX (US)

(21) Appl. No.: 18/003,744

(22) PCT Filed: Aug. 27, 2021
(86) PCT No.: PCT/US2021/048081

§ 371 Date: Dec. 29, 2022

Related U.S. Application Data
(60) Provisional application No. 63/046,611, filed on Jun. 30, 2020.

(51) Int. Cl. A61K 41/00; A61K 33/242; A61K 9/51; A61P 35/00; A61N 5/10

(52) U.S. Cl. CPC A61K 41/0038; A61K 33/242; A61K 9/51; A61P 35/00; A61N 5/10; A61N 2005/1087; A61N 2005/1098

(57) ABSTRACT

We disclose a method, comprising administering, to a patient suffering from a cancer, a composition comprising a compound containing a gold atom; and administering, to a portion of the patient's body in which the cancer is present, radiation. We also disclose a kit comprising a composition comprising a compound containing a gold atom; and instructions to perform the method.

### PRIORITY CLAIM

This application claims the right of priority to U.S. Provisional Patent Application 63/046,611, filed Jun. 30, 2020.

### FIELD OF THE INVENTION

The present invention relates generally to the field of cancer treatment. More particularly, it concerns the radiosensitization of cancer cells by in situ formation of gold nanoparticles or gold nanoclusters.

### GOVERNMENT SUPPORT STATEMENT

This invention was made with government support under grant number CA252156, awarded by the National Institutes of Health. The government has certain rights in the invention.

### BACKGROUND OF THE INVENTION

Radiation therapy (RT) is a long-established and effective component of modem cancer therapy for localized disease. However, the ultimate utility of radiation therapy is limited by the fact that some cancer cells are resistant to ionizing radiation. Additionally, the delivery of the ionizing radiation through healthy tissue or beyond the tumor margin limits the radiation dose and may result in unwanted side effects.

In recent years, intravenously administered nanoparticles (NPs) have shown great promise as anti-cancer agents. One of their potential uses has been radiation dose enhancement by particles made of high atomic number (Z) elements such as gold. Several studies have demonstrated radiation dose enhancement in the presence of gold nanoparticles (GNP) resulting in substantial tumor regression and long-term survival in tumor-bearing mice^{28,53-54} generating great excitement in the field of oncology. Unfortunately, enthusiasm for clinical translation of this strategy is dampened by (i) the high intratumoral GNP concentrations (˜1 mg/g tissue) needed, (ii) the strong dependence on the photon beam energy (kilovoltage (kV) x-rays), as predicted by Monte Carlo (MC) simulations, to achieve a significant (>10%) dose enhancement at a macroscopic scale, (iii) the requirement of almost simultaneous administration of GNPs and radiation, (iv) the lack of an understanding of underlying biological mechanisms driving the radiosensitization, and (v) the challenge of gaining entry of GNPs into tumor cells.

Pancreatic ductal adenocarcinoma (pancreatic cancer, PDAC) is the classic example of a recalcitrant tumor that is extremely challenging to treat. It is one of the most aggressive human malignancies, with a yearly incidence that equals its mortality.¹⁷ The only real chance for cure is surgical resection, but unfortunately only 15-20% individuals have resectable disease.¹⁸ Despite radical surgery, the overall survival rate for individuals with localized disease is approximately 20%. Administering systemic chemotherapy intravenously is limited by the hypovascularity and the dense stromal component (desmoplasia) of the tumor microenvironment.¹⁹⁻²⁴ These factors also contribute to a hostile microenvironment (low pH, low pO2) as well as presenting a physical barrier, “fencing” off the tumor from drugs or radiosensitizing agents. Therapeutic strategies, which can bypass the desmoplasia ‘fortress’ and apply therapy in hypoxic microenvironments without significantly affecting healthy cells and tissues would address the critical issues inherently presented by PDAC physiology.

Localized therapies are a critical component of treatment and there is renewed interest in innovative ways to intensify RT. The increased toxicity and lack of survival benefit from elective irradiation of locoregional nodal basins has led to a shift in the efforts towards focusing dose-escalation on just the primary tumor.²⁵ Stereotactic body radiation therapy (SBRT) complements this paradigm by allowing delivery of a highly conformal ablative dose over a relatively short period of time. In a recent phase II multi-institutional trial of SBRT in combination with single-agent gemcitabine showed overall survival (OS) of 13.9 months with low rates of acute and late grade >2 toxicities.²⁶ Other reports of fractionated SBRT suggest that OS of up to 15 months are achievable. SBRT has the advantage of requiring just 5 fractions of treatment and is usually performed by placing radiopaque fiducials in the tumor, a procedure that lends itself to co-opting for delivery of intratumorally injected agents (i.e., gold ions). The feasibility of intratumoral injection of a therapeutic agent before the first fraction of RT each week was established in a randomized study of TNFerade.²⁷ Despite these advances, there remains a critical need to develop new methods to increase dose delivery to cancer cells while minimizing damage to normal tissue for the best outcomes.

Ultimate utility of RT is limited by resistance of some cancer cells to the treatment. Attempts to improve outcomes of RT have largely focused on (i) increasing the dose of radiation delivered to the tumor, (ii) sensitizing the radioresistant fraction of tumor cells to conventional doses of RT, and (iii) targeting cancer cells specifically while administering RT. A novel approach to enhancing the radiation dose delivered to tumors is by transiently increasing the radiation-interaction probability of the target tissues using high-Z materials. A pioneering study showed a 66% increase in one-year survival for mammary tumor-bearing mice receiving radiotherapy after intravenous injections of 1.9 nm GNPs compared to mice without gold treatment.²⁸ This is attributed to an increase in photoelectric absorption interactions due to the high Z of gold followed by the greater physical damage to tumor cells and endothelial cells by photoelectrons from GNPs. However, the extremely large quantities of gold in tumors (7 mg/g), the timing of radiation (2 min after injection) and the radiation used (single 26 Gy dose of 250 kVp x-rays) in this study was clinically unappealing. Nonetheless, this initial demonstration laid the foundation for more extensive evaluation of a GNP-based radiosensitization. Subsequent studies demonstrated the possibility of potent radiosensitization even when the concentration of gold within tumors (˜0.0004 mg/g) is over a thousand-fold lower than that previously felt to be necessary.²⁹⁻³⁶ This improvement was achieved by increasing intracellular localization of GNPs using cancer cell specific targeting.

However, in the microenvironment of various types of cancers, including pancreatic cancer, even the smallest nanoparticles are diffusion limited by the desmoplasia, which prevents efficient delivery to cancer cells. Indeed, pancreatic cancer is characterized by hypovascularity in the setting of a dense stromal component with an exuberant interstitial matrix of glycosaminoglycans, collagen, and proteoglycans (desmoplasia) that serves as a physiological barrier to the delivery of drugs and nanoparticles. The consequent hostile microenvironment (low pH, low pO2) of the tumor core harbors the most aggressive tumor cells with the greatest potential to regenerate if they survive cytotoxic treatment. This problem is further amplified by the presence of gastrointestinal mucosa immediately adjacent to the tumor that makes dose escalation difficult and often not readily achievable.

Thus, there is a need for new, more effective, radiosensitization methods.

### SUMMARY OF THE INVENTION

The following presents a simplified summary of the invention in order to provide a basic understanding of some aspects of the invention. This summary is not an exhaustive overview of the invention. It is not intended to identify key or critical elements of the invention or to delineate the scope of the invention. Its sole purpose is to present some concepts in a simplified form as a prelude to the more detailed description that is discussed later.

In one embodiment, the present invention relates to a method, comprising administering, to a patient suffering from a cancer, a composition comprising a compound containing a gold atom; and administering, to a portion of the patient's body in which the cancer is present, radiation.

In one embodiment, the present invention relates to a kit, comprising a composition comprising a compound containing a gold atom; and instructions for use of the composition in a method comprising administering, to a patient suffering from a cancer, the composition; and administering, to a portion of the patient's body in which the cancer is present, radiation.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 presents a flowchart of a method in accordance with embodiments herein.

FIG. 2A depicts locally injected gold atoms uniformly distributed throughout a pancreatic cancer tumor due to their atomic size, in accordance with embodiments herein.

FIG. 2B depicts cancer specific biosynthesis of gold nanoparticles with nuclear localization, in accordance with embodiments herein.

FIG. 2C depicts sensitization of cancer cells to local radiation therapy with minimum off-target damage to normal cells, in accordance with embodiments herein

FIG. 3A presents fluorescence images of control NIH3T3 cells (left) and cells treated with Au³⁺ (right). Both control and treated cells generated fluorescence with nuclear staining by Hoechst 33342. Fluorescent signal in the treated cells was associated with intracellular formation of gold nanoclusters (GNCs). Scale bars 20 μm.

FIG. 3B reports cell viability after radiation relative to viability of control cells that were not exposed to gold at 0 Gy.

FIG. 4 presents fluorescence confocal images of non-cancerous (HPDE) and cancerous (MIAPaCa2) pancreatic cells treated with either 1 mM of Au³⁺ gold ions or premade albumin-GNCs at 1 mM Au⁰ for 24 hours. (From left to right) Cells: untreated; treated with albumin-GNCs; treated with Au³⁺ gold ions. The far-right image shows cells treated with Au³⁺ at a higher magnification. All cells showed blue fluorescence from Hoechst nuclear stain. Red color showing fluorescence signal from GNCs was ubiquitous in the MIAPaCa2 cells treated with Au³⁺ gold ions and was only sparsely seen in HPDE cells treated with Au³⁺ gold ions or albumin-GNCs. The strong red fluorescence in cancer cells indicated intracellular synthesis of GNCs from gold ions. Scale bars are 25 μm.

FIG. 5 shows cross-sections of confocal fluorescence images of MIAPaCa pancreatic cancer cells treated with Au³⁺showing localization of in situ-synthetized GNCs (center, “GNC”) inside nuclei (right, visualized by “Hoechst” stain). Nuclear boundaries are outlined in the merged image (left, “Merge”) for better visualization.

FIG. 6A shows live-cell confocal fluorescence images from x-ray irradiation of pancreatic cells, treated with either ionic gold (Au³⁺), or without treatment. Hoechst 33342 stain was used for nuclear contrast. Red channel was used for detection of GNCs with 610 nm emission and 561 nm laser excitation. Scale bars are 25 μm. We observed significant radiosensitization effect for treated cancerous cells (bottom right) and no radiosensitization for treated non-cancerous cells (top right) or untreated cells (top and bottom left).

FIG. 6B shows clonogenic assay results from x-ray irradiation of pancreatic cells, treated with either ionic gold (Au³⁺), or without treatment.

FIG. 7 shows fluorescence images of gold nanoclusters formed resulting from 24 hr treatments of 1.00 mM Au³⁺ (as chloroauric acid) in full cell media to PANC1 pancreatic cancer cells. Cells are live during imaging. Scale bars are 10 μm.

FIG. 8A shows fluorescence images of gold nanoclusters formed resulting from 24 hr. treatments of either 1.00 mM Au³⁺ (as chloroauric acid), Au⁰ pre-fabricated albumin coated gold nanoclusters of similar fluorescent properties, or without treatment in full cell media to HPDE Pancreatic cells (top row), MIAPACA2 Pancreatic cancer cells (middle row), and PANC1 pancreatic cancer cells (bottom row). Hoechst nuclear stain is the only imaging source in the untreated column and the primary imaging source in the albumin-GNC column and the non-cancer Au³⁺ panel. Cells are live during imaging. Scale bars are 10 μm.

FIG. 8B quantifies GNC channel pixel intensity of the samples in each of the rows of FIG. 8A.

FIG. 9A shows fluorescence images of gold nanoclusters formed resulting from 24 hr. treatments of 1.00 mM Au³⁺ (as chloroauric acid) in full cell media to PANC1 pancreatic cancer cells with Hoechst nuclear stain with cross sectional imaging demonstrating the gold nanocluster fluorescence is internal to the cell nuclei. Cells are live during imaging. Scale bars are 20 μm.

FIG. 9B shows transmission electron micrographs of PANC1 pancreatic cancer cells treated with 1.00 mM Au³⁺ (as chloroauric acid) in full cell media. Gold nanoparticles within the nucleolus can readily be seen in highest magnification view (right).

FIG. 10 presents fluorescence images of gold nanoclusters formed resulting from 24 hr. treatments of 1.00 mM Au³⁺ (as chloroauric acid) in full cell media to PANC1 pancreatic cancer cells with Hoechst nuclear stain, under varied concentrations of fetal bovine serum (FBS) in the growth media. Cells are live during imaging. Scale bars are 20 μm

FIG. 11 shows fluorescence images of gold nanoclusters formed resulting from 24 hr. treatments of 1.00 mM Au³⁺ (as chloroauric acid) in full cell media to PANC1 pancreatic cancer cells with Hoechst nuclear stain, under varied durations of time for cells to condition the growth media prior to treatment. Cells are live during imaging. Scale bars are 20 μm.

FIG. 12 presents fluorescence images of gold nanoclusters formed resulting from treatments of 1.00 mM Au³⁺ (as chloroauric acid) in full cell media to PANC1 pancreatic cancer cells with Hoechst nuclear stain, under varied treatment duration times. Cells are live during imaging. Scale bars are 20 μm.

FIG. 13 shows fluorescence images of gold nanoclusters formed resulting from 24 hr. treatments of Au³⁺ (as chloroauric acid) in full cell media to PANC1 pancreatic cancer cells with Hoechst nuclear stain, under varied treatment Au³⁺ treatment concentrations. Cells are live during imaging. Scale bars are 20 μm.

FIG. 14 graphs fluorescent nanoparticle formation (ex560/em610 nm) with plasmonic nanoparticle formation (A550 nm) as a function of Au³⁺ treatment concentration made over 24 hours in full cell media to PANC1 pancreatic cancer.

FIG. 15 reports cell viability as a function of 24 hour Au³⁺treatments at varied concentrations determined via AO/PI live-dead assay and in full cell media to PANC1 pancreatic cancer.

FIG. 16 shows fluorescent nanoparticle formation (ex560/em610 nm) as a function of Au³⁺ treatment concentration and cell density made over a 20 hour period in full cell media to PANC1 pancreatic cancer.

FIG. 17 shows plasmonic nanoparticle formation (A550 nm) as a function of Au³⁺ treatment concentration and cell density made over a 20 hour period in full cell media to PANC1 pancreatic cancer.

FIG. 18 shows longitudinal Pancl pancreatic cancer cell fluorescence across a 20 hr time period resulting from 0.20 mM treatment of Au³⁺ (as chloroauric acid) in full cell media.

FIG. 19 reports cell viability as a function of 24 hour Au³⁺ treatments at varied concentrations determined via JC-1 mitochondrial depolarization assay and in full cell media to PANC1 pancreatic cancer.

FIG. 20 presents evidence of radiosensitization resulting from 24 hour 0.20 mM Au³⁺ treatments (lower plot) compared against non-treated (upper plot) determined via clonogenic survival assay and in full cell media to PANC1 pancreatic cancer.

FIG. 21 reports on a mechanistic study of radiosensitization quantifying gamma H2AX foci through fluorescent antibody staining measured at 0, 4, and 24 hours, resulting from 24 hour 0.20 mM Au³⁺ treatments (lower three) compared against non-treated (upper three) combined with either 0 Gy or 8 Gy x-ray irradiation. Treatments are in full cell media to PANC1 pancreatic cancer.

FIG. 22 reports on a mechanistic study of radiosensitization quantifying mitochondrial depolarization through JC-1 assay measured at 0, 1, and 24 hours, resulting from 24 hour, 0.20 mM Au³⁺ treatments (lower three) compared against non-treated (upper three) combined with either 0 Gy or 8 Gy x-ray irradiation. Treatments are in full cell media to PANC1 pancreatic cancer.

FIG. 23 reports on a mechanistic study of radiosensitization quantifying total NADP through NADP assay measured at 0, 1, and 24 hours, resulting from 24 hour, 0.20 mM Au³⁺ treatments (right four bars) compared against non-treated (left four bars) combined with either 0 Gy or 8 Gy x-ray irradiation. Treatments are in full cell media to PANC1 pancreatic cancer.

FIG. 24 reports on a mechanistic study of radiosensitization quantifying the ratio of NADP⁺/NADPH through NADP assay measured at 0, 1, and 24 hours, resulting from 24 hour, 0.20 mM Au³⁺ treatments (right) compared against non-treated (left) combined with either 0 Gy or 8 Gy x-ray irradiation. Treatments are in full cell media to PANC1 pancreatic cancer.

FIG. 25 reports on a mechanistic study of radiosensitization quantifying the ratio of peroxidation product formation resulting from X-ray damage through TBARS assay, resulting from 24 hour, 0.20 mM Au³⁺ treatments (right) compared against non-treated (left) combined with either 0 Gy or 8 Gy x-ray irradiation. Treatments are in full cell media to PANC1 pancreatic cancer.

FIG. 26 presents evidence of radiosensitization, quantifying the cell viability resulting from X-ray damage through MTT assay measured 24 and 96 hours after x-ray irradiation, resulting from 24 hour, 0.20 mM Au³⁺ treatments (right bar in each dosage pair) compared against non-treated (left bar in each dosage pair) combined with either 0 Gy or 8 Gy x-ray irradiation. Treatments are in full cell media to PANC1 pancreatic cancer.

FIG. 27A. Fluorescence nanoparticle formation through IVIS imaging (ex610/em660 nm) of nanoparticle formation in PANC1 xenografts in nu/nu mice 48 hours after treatment with 1.00 mM Au³⁺ (as chloroauric acid).

FIG. 27B. Fluorescence of extracted organs of treated mice shown in FIG. 27A.

FIG. 28A shows transmission electron micrographs of nanoparticle formation in PANC1 xenografts in nu/nu mice 48 hours after treatment with 1.00 mM Au³⁺ (as chloroauric acid).

FIG. 28B quantifies particle diameters from the transmission electron micrographs shown in FIG. 28A.

FIG. 29 shows blood chemistry and hematology data following nanoparticle formation in PANC1 xenografts in nu/nu mice 48 hours after treatment with 1.00 mM Au³⁺ (as chloroauric acid) vs. controls.

FIG. 30 shows blood chemistry and hematology data following nanoparticle formation in PANC1 xenografts in nu/nu mice 48 hours after treatment with 1.00 mM Au³⁺ (as chloroauric acid) vs. controls.

FIG. 31 shows blood chemistry and hematology data following nanoparticle formation in PANC1 xenografts in nu/nu mice 48 hours after treatment with 1.00 mM Au³⁺ (as chloroauric acid) vs. controls.

FIG. 32 shows evidence of radiosensitization effect from nanoparticle formation in PANC1 xenografts in nu/nu mice 48 hours after treatment with 1.00 mM Au³⁺ (as chloroauric acid) (bottom and uppermost traces) compared to non-treated (middle two traces) by tumor volume measurements occurring after 10 Gy X-ray irradiation.

FIG. 33 shows fluorescence images of gold nanoclusters formed resulting from 24 hr. treatments of Au³⁺ (as chloroauric acid) in full cell media to 8505C thyroid cancer cells and Nthy-Ori-3-1 normal thyroid cells with Hoechst nuclear stain (blue), under varied treatment Au³⁺ treatment concentrations. Cells are live during imaging. Scale bars are 20 μ.

FIG. 34 shows fluorescence images of gold nanoclusters formed resulting from 24 hr. treatments of 1.00 mM Au³⁺ (as chloroauric acid) in full cell media to 8505C thyroid cancer cells with Hoechst nuclear stain with cross sectional imaging demonstrating the gold nanocluster fluorescence is internal to the cell nuclei. Cells are live during imaging. Scale bars are 20 μm.

FIG. 35A. Darkfield images of gold nanoparticle formation resulting from 24 hr. treatments of 1.00 mM Au³⁺ (as chloroauric acid) in full cell media to 8505C thyroid cancer and Nthy-Ori-3-1 normal thyroid cells with Hoechst nuclear stain. Cells are fixed for imaging. Scale bars are 20 μm.

FIG. 35B. Darkfield intensity areas under the curve (AUCs) for the images shown in FIG. 35A.

FIG. 36 shows cell viability as a function of 24 hour Au³⁺ treatments at varied concentrations determined via MTT assay and in full cell media to 8505C thyroid cancer and Nthy-Ori-3-1 normal thyroid cells.

FIG. 37 shows evidence of radiosensitization via induced double stranded DNA breaks in thyroid cancer quantifying gamma H2AX foci through fluorescent antibody staining measured at 24 hours after x-ray irradiation, resulting from 24 hour treatments of 0.20 mM of either Au³⁺ or Au⁰ prefabricated gold particles (GNPs) compared against non-treated combined with either 0 Gy or 8 Gy x-ray irradiation. Treatments are in full cell media.

While the subject matter disclosed herein is susceptible to various modifications and alternative forms, specific embodiments thereof have been shown by way of example in the drawings and are herein described in detail. It should be understood, however, that the description herein of specific embodiments is not intended to limit the invention to the particular forms disclosed, but on the contrary, the intention is to cover all modifications, equivalents, and alternatives falling within the spirit and scope of the invention as defined by the appended claims. Moreover, the stylized depictions illustrated in the drawings are not drawn to any absolute scale.

### DESCRIPTION OF ILLUSTRATIVE EMBODIMENTS

Various illustrative embodiments of the invention are described below. In the interest of clarity, not all features of an actual implementation are described in this specification. It will of course be appreciated that in the development of any such actual embodiment, numerous implementation-specific decisions must be made to achieve the developers' specific goals, such as compliance with system-related, regulatory, and business-related constraints, which will vary from one implementation to another. Moreover, it will be appreciated that such a development effort might be complex and time-consuming but would nevertheless be a routine undertaking for those of ordinary skill in the art having the benefit of this disclosure.

The present subject matter will now be described with reference to the attached figures. Various structures, systems, and devices are schematically depicted in the drawings for purposes of explanation only and so as to not obscure the present disclosure with details that are well known to those skilled in the art. Nevertheless, the attached drawings are included to describe and explain illustrative examples of the present disclosure. The words and phrases used herein should be understood and interpreted to have a meaning consistent with the understanding of those words and phrases by those skilled in the relevant art. No special definition of a term or phrase, i.e., a definition that is different from the ordinary and customary meaning as understood by those skilled in the art, is intended to be implied by consistent usage of the term or phrase herein. To the extent that a term or phrase is intended to have a special meaning, i.e., a meaning other than that understood by skilled artisans, such a special definition will be expressly set forth in the specification in a definitional manner that directly and unequivocally provides the special definition for the term or phrase.

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, any given numerical value includes the inherent variation of error for the device, or the method being employed to determine the value, or the variation that exists between study subjects or healthcare practitioners.

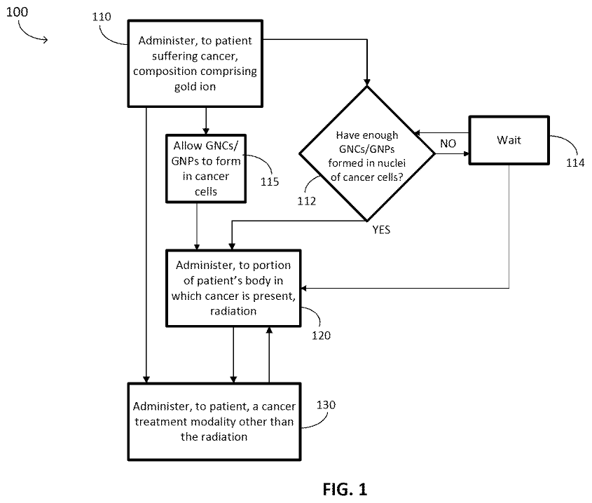

FIG. 1 presents a flowchart of a method 100 in accordance with embodiments of the present disclosure. The method 100 comprises administering 110, to a patient suffering from a cancer, a composition comprising a compound containing a gold atom; and administering 120, to a portion of the patient's body in which the cancer is present, radiation.

The patient may be any mammal suffering from the cancer. In one embodiment, the patient is a human being.

In embodiments, the present method may be performed in a veterinary context. That is, the patient may be any non-human mammal suffering from a cancer. The non-human mammal may be a research animal, a pet, livestock, a working animal, a racing animal (e.g., a horse, a dog, a camel, etc.), an animal at stud (e.g., a bull, a retired racing stallion, etc.), or any other non-human mammal for which it is desired to treat its cancer.

For convenience, the description will typically refer to human patients. However, the person of ordinary skill in the art having the benefit of the present disclosure will readily be able to adapt the teachings of the present disclosure to a veterinary context.

By “suffering from a cancer” is meant that the cancer is detectable in the patient's body using any diagnostic technique presently known or to be discovered. “Suffering” does not require the patient to be in pain from or have any naturally-perceptible symptoms of the cancer. Generally, as is known, the earlier a cancer can be treated, including before the patient notices pain or any other symptoms, the greater the chances of remission.

The present method may be used to treat any type of cancer. Desirably, the cancer is one that is known or reasonably expected, by the person of ordinary skill in the art having the benefit of the present disclosure, to be treatable by radiation after radiosensitization by gold.

In one embodiment, the cancer is characterized by a desmoplastic stroma. The stroma is a biological structure containing one or more of connective tissue, blood vessels, and inflammatory cells in the cancer microenvironment. Desmoplastic stroma is stroma that is dense and fibrous. One comment characteristic of desmoplastic stroma is limited delivery of therapeutic molecules to tumor cells. In one embodiment, the desmoplastic stroma may limit diffusion of particles having a minimum dimension of 5 nm or greater to malignant cells of the cancer. By “limits diffusion” is meant that the rate of in vivo uptake of the particles by the malignant cells is reduced for the cells that are located further away from the blood vessels or injection site, i.e., the bigger the particle, the fewer particles reach malignant cells. Furthermore, the denser the stroma, the fewer particles diffuse inside the tumor and the fewer the particles delivered to malignant cells.

Not every presentation of the type of the cancer must feature a stroma having this diffusion-limiting parameter for the cancer to be “characterized by a desmoplastic stroma.”

By “minimum dimension” is meant the diameter, for spheres, or the maximum width of the smallest dimension, for oval or approximately rectangular or spherical particles.

In one embodiment, the cancer is selected from the group consisting of pancreatic cancer, head-and-neck cancer, anaplastic thyroid cancer, brain cancer, liver cancer, and breast cancer. These cancers are well-recognized as being characterized by a dense stroma However, the method 100 may be performed on presentations of these cancers which are not characterized by a dense stroma.

In one particular embodiment, the cancer is pancreatic cancer.

In another embodiment, the cancer is head-and-neck cancer.

In yet another embodiment, the cancer is anaplastic thyroid cancer.

In an additional embodiment, the cancer is brain cancer.

In yet an additional embodiment, the cancer is liver cancer.

In an embodiment, the cancer is breast cancer.

The composition to be administered 110 comprises a compound containing a gold atom. By “compound containing a gold atom” is meant a compound containing gold in any oxidation/reduction state. The gold atom may be present as individual atoms, soluble salts, or as part of a molecule, polymer, or multiatom ion. The compound may contain one or more other atoms in any redox state that are one or more of covalently bound to a gold atom, ionically paired with a gold atom, or otherwise associated with a gold atom. In one embodiment, the compound may be an ionic compound containing an ion, typically an anion (a negatively charged ion) comprising gold in the Au' oxidation state, and a cationic counterion (positively charged ion), such as sodium, hydrogen, or another cation known for use in pharmaceutical salts and ionic compounds.

Use of the singular term “a compound” does not limit the composition to comprising only one compound containing a gold atom. The singular term “a gold atom” does not limit the compound(s) to comprising only one gold atom.

In one embodiment, the compound containing a gold atom is selected from those disclosed by C. Frank Shaw III, “Gold-Based Therapeutic Agents,” Chem Rev 1999, hereby incorporated herein by reference.

In one embodiment, the compound containing a gold atom is selected from the group consisting of triethylphosphine(2,3,4,6-tetra-O-acetyl-β-1-d-thiopyranosato-S)gold(I), aurothioglucose salts, auranofin salts, aurothiomalate salts, chloroaurate salts, buffered chloroauric acid, (Φ₃PAu)₂(μDTE), Φ₃PAutTP, Φ₃PAu-thymidine, Φ₃PAu(5-fluorouridine), Φ₃PAu(tegafur), ferrocene(μ-Φ₂PAuCl)₂, Et₃PAuCl, Et₃PAuCN, Et₃PAuCH₃, [(Et₃P)₂Au]Cl, Et₃PAuSCN, Et₃PAuSCH₃, Et₃PAuSG, Et₃PAuSTg, Et₃PAuSAtg (auranofin), Et₃PAuS-α-Atg (epiauranofin), [AuSTm]ₙ, [AuSTg]ₙ, [AuSATg]ₙ, DPPE(AuCl)₂, DPPE(AuSTg)₂, [Au(DPPE)₂]Cl, [Au(R2P-Y-PR'₂)₂]X, Au(Streptonigrin), [Me₂AuCl₂][AsΦ₄], Me₂Au(μSCN)₂AuMe₂, Au(N-methylimidazole)Cl₃, Au(2-methylbenzoxazole)Cl₃, Au(2,5-dimethylbenzoxazole)Cl₃, DPPE(AuCl₃)₂, [Au(damp)Cl₂], [Au(damp)(SCN)₂], [Au(damp)(OAc)₂], [Au(damp)(malonate)], ⁱPr₃PAuCN, Ph₃PAuCN, Cy₃PAuCN, KAu(CN)₂, AuCl₄⁻, Au³⁺, Au¹⁺, and mixtures thereof

In one embodiment, the compound containing a gold atom is selected from the group consisting of triethylphosphine(2,3,4,6-tetra-O-acetyl-β-1-d-thiopyranosato-S)gold(I), aurothioglucose salts, auranofin salts, aurothiomalate salts, chloroaurate salts, buffered chloroauric acid, and mixtures thereof

In a particular embodiment, the compound containing a gold atom is selected from chloroaurate salts.

The concentration of the compound containing a gold atom may be varied depending on the route of administration, the presence or absence of other compounds in the composition, and other factors. The concentration may be selected as a routine matter by the person of ordinary skill in the art having the benefit of the present disclosure.

In one embodiment, the administering 110 the composition comprises administering to the patient an amount of gold from 0.0001 mg/g tumor cells to 10 mg/g tumor cells. The mass of tumor cells generally cannot be precisely weighed, but the person of ordinary skill in the art may generally

The composition may also comprise a solvent in which the compound containing a gold atom may be dissolved. Conveniently, the solvent may be water, although other hydrophilic or polar solvents that are pharmaceutically-acceptable may be used.

In one embodiment, the composition may further comprise one or more other pharmaceutically-acceptable compounds known for use in solution medicaments, such as buffers, preservatives, adjuvants, surfactants, diluents (e.g. saline or dextrose) or the like. Such particular other compounds may be routinely selected by the person of ordinary skill in the art having the benefit of the present disclosure.

Though not to be bound by theory, we have observed that compounds containing a gold atoms are generally preferentially taken up by cancer cells relative to normal cells. Accordingly, the composition generally lacks a need for targeting molecules or moieties.

Though not to be by theory, we have observed that compound containing a gold atoms, after being taken up by cancer cells, tend to form gold nanoclusters (GNCs) and/or gold nanoparticles (GNPs) in situ. By “gold nanoclusters” is meant agglomerations comprising gold. By “gold nanoparticles” is meant gold nanoclusters that have a minimum dimension of 1 nm or greater. The gold nanoparticles and gold nanoclusters are not limited to any particular shape or structural motif. Gold nanoparticles formed in situ may have a minimum dimension of 5 nm or greater, i.e., if pre-formed outside the cancer cell, would undergo limited diffusion through the stroma. Further, though again not to be bound by theory, we have observed that in situ GNC/GNP formation tends to occur in the cancer cell nucleus. From this, the person of ordinary skill in the art would expect that radiation dose enhancement arising from the in situ GNC/GNPs would inflict more damage on DNA and other structures in the cancer cell nucleus than in other structures of the cancer cell and would inflict more damage on those other structures of the cancer cell than on normal cells in the vicinity.

In one embodiment, the composition may comprise a micelle, liposome, a mesoporous silica particle, a polymersome, a polyethylene glycol (PEG) polymer cluster, a tri-block amphiphilic polymer, a di-block amphiphilic polymer, or two or more thereof. In a further embodiment, the composition may additionally comprise a moiety which preferentially interacts with one or more tumor-related targets.

Alternatively, or in addition, the composition may comprise one or more release extension agents. For example, micelles, liposomes, mesoporous silica particles, polymersomes, PEG polymer clusters, and di- and tri-block amphiphilic polymers, among others, may allow extended release of gold atoms or ions. By the inclusion of such agents, the release from the composition of the compound containing a gold atom, or gold atoms or ions themselves, may proceed at a relatively steady rate for an extended period of time.

The composition may be administered 110 to the patient by any route. Such routes may be characterized as systemic or local. Systemic routes include oral, nasal, buccal, and intravenous injection routes, among others. Local routes include subcutaneous, intramuscular, intraorganal, and intratumoral injection, and catheterized and endoscopic routes, among others. Generally, local routes in proximity to malignant cells of the cancer may be desirable, in that they are expected to require lower doses of the compound containing a gold atom, may reduce the risk of side effects, and may lead to more ready uptake of the compound containing a gold atom, or the gold atom itself, by the cancer cells.

In one embodiment, administering 110 the composition comprises injection of the composition in proximity to malignant cells of the cancer.

In the method 100, administering 110 the composition may be performed in a single dose or a plurality of doses. A plurality of doses may be desirable if the total amount of gold to be delivered would have toxic effects on healthy tissue if delivered in a single dose. If a plurality of doses is performed, the number of doses and the time between doses can be selected as a routine matter by the person of ordinary skill in the art having the benefit of the present disclosure.

The method 100 also comprises administering 120, to a portion of the patient's body in which the cancer is present, radiation.

Radiation therapy is a well-known cancer therapy technique. Generally, radiation comprising particles or photons that have sufficient energy or can produce sufficient energy via nuclear interactions is aimed at cancer cells to produce ionization (i.e., loss of electrons) in the cancer cells. This ionization generates reactive oxygen species, which can damage cellular structures directly, or may damage DNA, thereby disrupting transcription and translation and thereby disrupting cellular function. Exemplary ionizing radiation types include X-ray radiation and proton radiation. Apparatus and techniques for delivering X-rays or protons to a target tissue or cell are well known in the art.

The amount of ionizing radiation needed in a given cell generally depends on the nature of that cell. Means for determining an effective amount of radiation are well known in the art. For example, dosage ranges for X-rays range from daily doses of 50 to 200 cGy for prolonged periods of time (3 to 8 weeks), to single or a small number (3-5) doses of 500 to 2500 cGy. Common, but not limiting, X-ray treatment protocols involve five doses, one each on consecutive days or on alternating days.

In one embodiment, the administering 120 the radiation comprises administering X-rays or protons. In one particular embodiment, the administering 120 the radiation comprises administering X-rays. In another particular embodiment, the administering 120 the radiation comprises administering protons.

After administering 110 the composition, it may be desirable to allow time for the gold atom or the compound containing a gold atom to penetrate the stroma, be taken up by the cancer cells, and form in situ GNC/GNPs. Accordingly, in one embodiment, the method 100 further comprises allowing 115 gold nanoclusters (GNCs) and/or gold nanoparticles (GNPs) to form in the cancer cells. Because in situ GNC/GNP formation in cancer cells, especially pancreatic cancer cells, is spontaneous, no further action is required. In one embodiment, administering 120 the radiation is performed from 0 seconds to 14 days after administering 110 the composition. In one embodiment, administering 120 the radiation may be performed from 30 minutes to 24 hours after administering 110 the composition. In embodiments wherein administering 110 the composition is performed in multiple doses, administering 120 the radiation is performed from 0 seconds to 14 days after the final dose of the composition. In particular embodiments, administering 120 the radiation may be performed from 30 minutes to 24 hours after administering 110 the final dose of the composition.

Generally, in situ formation of GNC/GNPs is expected after administering 110 the composition. However, depending on the cancer, the type of radiation, the patient's sensitivity to radiation, and/or other parameters, it may be desirable to detect GNC/GNPs formed in situ after administering 110 the composition. In one embodiment, the method 100 may further comprise determining 112, after the administering the composition, whether an amount of GNC/GNPs, sufficient for radiation dose enhancement have formed in the nuclei of one or more malignant cells of the cancer. For example, determining 112 may comprise extracting malignant cells of the cancer from the patient's body and observing GNC/GNP by confocal fluorescence microscopy, flow cytometry, or other techniques that will be known to the person of ordinary skill in the art. Determining whether the amount of GNC/GNPs is sufficient for radiation dose enhancement will depend on one or more of the total mass of gold in the GNC/GNPs, the shape and structure of the GNC/GNPs, the proximity of the GNC/GNPs to the cancer cell nucleus, the type of cancer cell, or the nature and intended dosage of the radiation, among other parameters that will be apparent to the person of ordinary skill in the art having the benefit of the present disclosure.

If determining 112 is performed, and the outcome is that an insufficient amount of GNC/GNPs have formed, the method 100 flows to a wait 114. After the wait 114, flow may return to determining at 112, or it may be presumed that enough GNC/GNPs have formed, and flow may pass to administering 120 the radiation.

The method 100 may comprise additional events. In one embodiment, the method 100 may further comprise administering 130, to the patient, a cancer treatment modality other than the radiation. Administering 130 the cancer treatment modality other than the radiation may be targeted against the same cancer as the radiation, against metastases thereof, against a primary tumor or metastases of a cancer other than cancer targeted by the radiation, or two or more thereof

A wide variety of cancer treatment modalities other than radiation are known to the person of ordinary skill in the art and need not be described in detail here. By way of example, in one embodiment, the cancer treatment modality other than the radiation is selected from the group consisting of surgical resection, chemotherapy, immunotherapy, checkpoint inhibitor therapy, oncolytic virus therapy, thermal therapy (e.g., RFA, microwave ablation, and/or cryotherapy), and two or more thereof

Regardless of the particular cancer treatment modality other than radiation, if one or more is/are administered 130, the administering 130 may be performed before, after, or simultaneously with the administering 120 the radiation. Particular relative and absolute timing of administering 120 the radiation and administering 130 the other cancer treatment modality will be a routine matter for the person of ordinary skill in the art having the benefit of the present disclosure.

In one embodiment, the present disclosure relates to a kit, comprising a composition comprising a compound containing a gold atom; and instructions for use of the composition in a method comprising administering, to a patient suffering from a cancer, the composition; and administering, to a portion of the patient's body in which the cancer is present, radiation.

A “kit,” as used herein, refers to a package containing the composition, and instructions of any form that are provided in connection with the composition in a manner such that a clinical professional will clearly recognize that the instructions are to be associated with the composition.

“Instructions” typically involve written text or graphics on or associated with packaging of compositions of the invention. Instructions also can include any oral or electronic instructions provided in any manner. Written text or graphics may include a website URL or a QR code encoding a website URL, where other instructions or supplemental information may be provided in electronic form.

The kit may contain one or more containers, which can contain the composition or a component thereof. The kits also may contain instructions for mixing, diluting, or administering the composition. The kits also can include other containers with one or more solvents, surfactants, preservatives, and/or diluents (e.g., normal saline (0.9% NaCl), or 5% dextrose) as well as containers for mixing, diluting, or administering the composition to the patient in need of such treatment.

The composition may be provided in any suitable form, for example, as a liquid solution or as a dried material. When the composition provided is a dry material, the material may be reconstituted by the addition of solvent, which may also be provided by the kit. In embodiments where liquid forms of the composition are used, the liquid form may be concentrated or ready to use.

The kit, in one embodiment, may comprise a carrier being compartmentalized to receive in close confinement one or more containers such as vials, tubes, and the like

The composition is described above. In one embodiment, the compound containing a gold atom is selected from the group consisting of triethylphosphine(2,3,4,6-tetra-O-acetyl-β-1-d-thiopyranosato-S)gold(I), aurothioglucose salts, auranofin salts, aurothiomalate salts, chloroaurate salts, buffered chloroauric acid, and mixtures thereof

The method is described above. In one embodiment, the instructions comprise instructions to administer the composition by injection of the composition in proximity to malignant cells of the cancer. Alternatively, or in addition, in one embodiment, the instructions comprise instructions to administer the radiation by administering X-rays or protons. Again, alternatively or in addition, in one embodiment, the instructions further comprise instructions to administer, to the patient, a cancer treatment modality other than the radiation.

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

Example 1

Specific Aims

Radiation therapy (RT) is an integral component of modern therapy for locally advanced unresectable pancreatic cancers. However, its ultimate utility is severely limited by the fact that some cancer cells are resistant to RT. Delivering higher doses of RT to the gross tumor to overcome radiation resistance has historically been challenging due to the limited radiation tolerance of the surrounding organs. Sequestering gold nanoparticles (GNPs) within tumors to amplify radiation-induced secondary electron showers has gained traction in recent years as a means to escalate radiation dose in the immediate vicinity of the nanoparticle thus confining the higher dose to the tumor and sparing surrounding tissues. However, pancreatic cancer is characterized by hypovascularity in the setting of a dense stromal component with an exuberant interstitial matrix of glycosaminoglycans, collagen, and proteoglycans (desmoplasia) that serves as a physiological barrier to the delivery of drugs and nanoparticles¹⁻³. The consequent hostile microenvironment (low pH, low pO2) of the tumor core harbors the most aggressive tumor cells with the greatest potential to regenerate if they survive cytotoxic treatment.⁴ This problem is further amplified by the presence of gastrointestinal mucosa immediately adjacent to the tumor that makes dose escalation difficult and often not readily achievable.

Here we propose to overcome problems with specific radiosensitization of pancreatic cancer cells in the context of a dense stromal environment by intratumoral delivery of an aqueous solution of the compound containing gold atoms (i.e., buffered chloroauric acid) instead of gold nanoparticles (GNP) thus achieving the ultimate reduction in size of a therapeutic agent—an atomic scale. Our hypothesis is that small compounds containing a gold atoms (i) will uniformly distribute throughout the tumor as their diffusion is not likely to be impeded by the stroma, and (ii) will be reduced to GNPs after specific uptake by cancer cells that (iii) will result in cancer cell radiosensitization to RT. This hypothesis is based on our compelling preliminary data demonstrating efficient synthesis of GNPs inside pancreatic cancer cells with a high nuclear localization that is critical for efficient radiosensitization due to a higher dose delivery to nuclei by the secondary Auger electrons. Furthermore, normal pancreatic cells did not significantly produce GNPs. In addition, recent literature reports demonstrated intracellular synthesis of GNPs from chloroauric acid⁵⁻¹⁴ occurs with higher efficiency in cancerous versus non-cancerous cells^{6,8,9,11,12} with a preferential nuclear localization of the nanoparticles.⁵⁻⁷ These studies further support our hypothesis of cancer specific intracellular synthesis of GNPs. Changing the current delivery paradigm from pre-made GNPs with sizes of 5-200 nm to delivery of ˜0.3 nm compounds containing gold atoms is associated with a staggering ˜16 to 1,400 size reduction of a gold therapeutic agent that is of paramount importance in penetrating desmoplastic tumors. Indeed, soluble compounds containing gold atoms are on the same size scale with similar transport kinetics as physiological salts (e.g., Ca2+, Na+, K+) which can diffuse even inside dense biological environments. Moreover, compounds containing gold atoms have decades-long history of a safe clinical use in treatment of rheumatoid arthritis¹⁵ providing a clear path towards clinical translation.

We envision clinical implementation of our approach as an added boost to significantly increase efficacy of stereotactic body radiotherapy (SBRT) in patients with a pancreatic tumor. Recent clinical data from our group and others shows that radiation dose enhancement increases local control and overall survival of locally advanced pancreatic cancer patients¹⁶. However, the proximity of gastrointestinal mucosa to the tumor in many instances precludes this dose escalation in clinical practice. But, when high atomic number (gold, hafnium) nanoparticles are present within tumors, irradiation of the tumor results in radiation dose enhancement via an increase in the fluence of photo-/Auger electrons ejected from gold/hafnium. We expect that changing the current paradigm from delivery of pre-made GNPs to in situ synthesis of GNPs by cancer cells will overcame delivery barriers in pancreatic tumors and, thus, will result in a highly significant improvement of RT outcomes. Here, we will test our hypothesis via two Specific Aims.

Aim 1. Optimization and characterization of intracellular synthesis of GNPs by pancreatic cancer cells.

1.1. Optimize the dose of the compound containing gold atoms and the timeframe for intracellular synthesis of GNPs. Compare efficiency of the GNP synthesis by normal and cancer cells.

1.2. Determine intracellular distribution of GNPs as a function of time. These studies will provide insight into mechanisms of intracellular synthesis and of intranuclear accumulation of GNPs.

Aim 2: Evaluate radiosensitization efficacy of in situ synthetized GNPs in models of pancreatic cancer.

2.1. Compare RT of cancer and normal cells after treatment with compounds containing gold atoms in vitro.

2.2 Determine toxicity of administration of compounds containing gold atoms in a murine model.

2.3. Determine in vivo biodistribution and cellular internalization of GNPs after intratumoral delivery of compounds containing gold atoms.

2.4. Determine radiosensitization efficacy and tumor distribution of in situ synthetized GNPs in an orthotopic human pancreatic patient derived xenograft murine tumor model.

These studies will provide the framework for continued development of a readily deployable radiosensitization strategy for pancreatic cancer. This strategy is inherently simplistic, with a single active component—gold atoms, but it takes advantage of a complex cell biology in order to produce therapeutic GNPs that localize to the nucleus.

Innovation

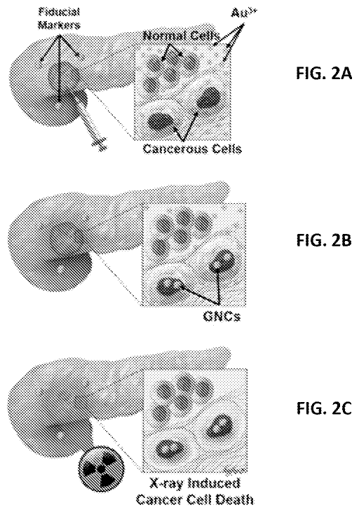

The key innovation of our approach is (1) a paradigm shift from delivery of pre-made GNPs to an atomic size gold precursor for tumor radiosensitization; this represents the ultimate size reduction of a therapeutic agent outside of the radiation therapy itself (i.e., x-rays, protons, etc.). Our strategy is inherently simplistic in design, as it employs a single, readily-procurable component (e.g., chloroauric acid) (FIG. 2A-2C). However, it also relies on a complex cell biology that is behind in situ synthesis of GNPs which is still poorly understood. We appreciate that the specificity of RT with our approach is dependent on differences in synthesis efficiency and in intracellular localization of GNPs in normal and cancer cells. Therefore, the second innovative aspect of our project is (2) studies of intracellular formation of GNPs in different cell types with an emphasis on gaining further understanding of cellular uptake, intracellular reduction, and trafficking of gold atoms by cancerous and normal cells. This understanding will provide foundation for future clinical applications of our strategy wherein radiosensitization agents are generated within the pathological tissue in a phenotype dependent manner as a cell-level personalized therapy. (3) Here this new concept will be validated in the specific context of PDAC. A dense desmoplasia is a signature of pancreatic cancer forming a formidable therapy delivery challenge that we plan to overcome with the ultimate size reduction of radiosensitizing precursors to an atomic level. Taken together these innovations will provide a clinically translatable solution to three key challenges in delivery of radiosensitization agents to PDAC: (i) tumor penetration, (ii) cancer specific cellular uptake and (iii) nuclear localization for efficient tumor radiosensitization that requires greatly reduced gold amount and more clinically relevant radiation (megavoltage radiation) than prior approaches to radiosensitization with GNPs.

Preliminary Data

Feasibility of radiosensitization via intracellular GNP formation

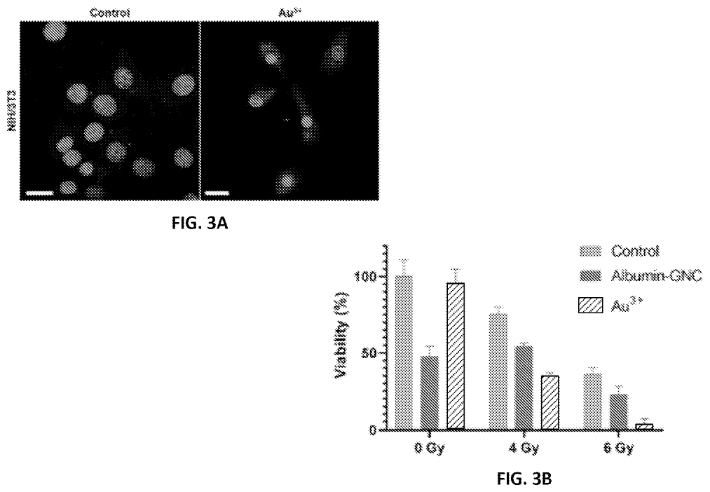

Our initial evaluation of radiosensitization via intracellular GNP formation was performed with 3T3 mouse fibroblast cells. 3T3 cells were chosen because they were previously characterized for intracellular GNP synthesis,¹¹ and fibroblasts are considered “bad players” and potential therapeutic targets in the pancreatic cancer microenvironmental niche.^{44,45} GNPs with sizes below 2 nm—gold nanoclusters (GNCs)—are known to exhibit a bright fluorescence in the visible region.¹¹ Therefore, their intracellular formation was verified via confocal fluorescence imaging with 561 nm excitation and 610 nm emission after cell treatment with 1 mM Au3+ (i.e., chloroauric acid) in cell culture media for 24 hours (FIG. 3A-3B). The images in FIG. 3A show uniform formation of GNCs in NIH3T3cells. Radiosensitization with intracellular GNC formation (i.e., delivery of Au³⁺) was compared with pre-fabricated albumin-coated GNCs (Albumin-GNC) prepared according to work by Xie⁴⁶ and control cells without treatment via MTS assay (FIG. 3B). Albumin-GNCs were chosen for comparison to see if a simple combination of extracellular gold nanoclusters with the most abundant serum protein (i.e., albumin) would produce a comparable radiosensitization to the intracellular synthetized GNCs.

3T3 cells were first incubated with either 1 mM of sodium chloroaurate or albumin-GNCs (0.1 mM Au⁰)in cell culture media for 10 hours. Then, the cells including the nontreated control were irradiated with X-rays at dosages of 0, 4 and 6 Gy in the X-ray X-RAD 225 CX irradiator system (Precision). The MTS assay showed a significant increase in radiosensitization by in situ synthetized GNCs as compared to Albumin-GNC control at both the 4 and 6 Gy doses (FIG. 3B).

In our future studies we will use a standard clonogenic survival assay for quantitation of the radiosensitization effect. Note that no difference in cell viability was observed between Au³⁺ treated and untreated cells at 0 Gy indicating that the incubation with chloroauric acid is not cytotoxic.

In situ synthesis of GNCs is greatly enhanced in pancreatic cancer cells as compared to non-cancerous cells

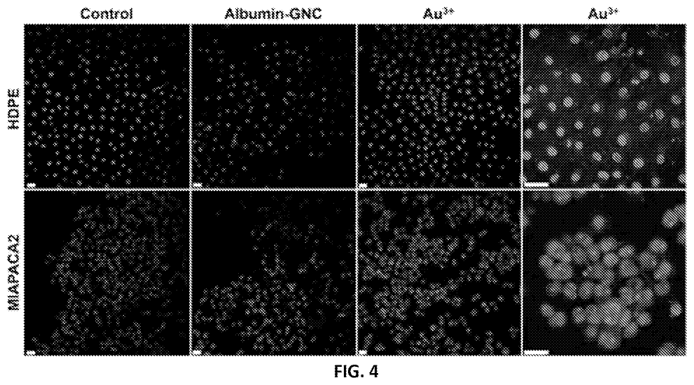

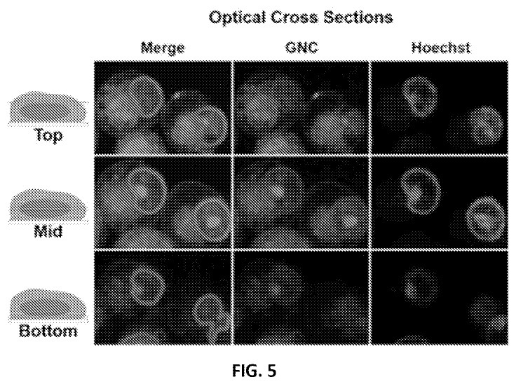

We compared in situ synthesis of GNCs by pancreatic cancer cells (MIAPACA2) and pancreatic noncancerous cells (HPDE) (FIG. 4). Confocal fluorescence images were obtained using a Leica TCS SP8 confocal microscope with 561 nm excitation and 610 nm emission optimized for detection of GNCs. Live cell nuclear stain (Hoechst 33342 was used to define location of nuclei. The fluorescence images reveal a striking increase in GNC formation in cancerous cells as compared to noncancerous cells after incubation with sodium chloroaurate (Au³⁺) (FIG. 4). Virtually no fluorescence was observed in either cell type incubated with pre-made albumin-GNCs indicating limited uptake of the extracellularly formed nanoclusters. In cancer cells (MIAPACA2), the fluorescence from GNCs was uniformly distributed throughout the intracellular space with a significant fraction in nuclei as revealed by optical sectioning (FIG. 5). Almost no background fluorescence was observed in the extracellular space (FIG. 4), indicating that GNCs are not synthetized in the extracellular environment.

In Situ Synthesis of GNCs is more prevalent in cancer cells with greater radiosensitization as compared to non-cancerous cells

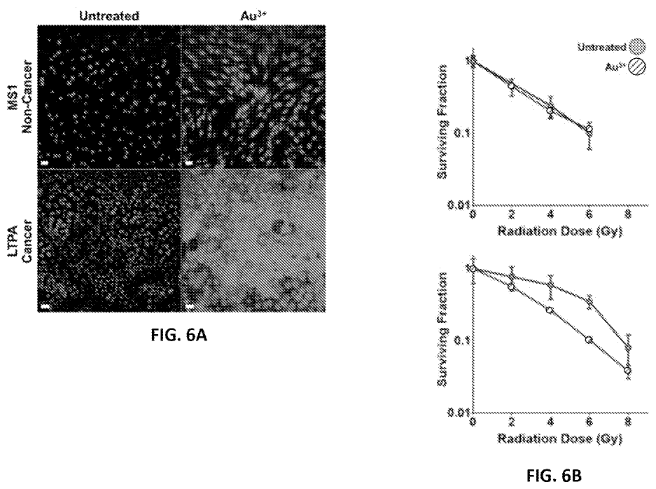

Our initial evaluation of intracellular gold nanocluster formation was performed with pancreatic cancer cells (LTPA) and pancreatic non-cancerous cells (MS1) (FIG. 6A-6B). Intracellularly formed GNCs are known to exhibit a bright fluorescence in the visible region.¹¹ Therefore, their intracellular formation was verified via confocal fluorescence imaging using Leica TCS SP8 confocal microscope with 561 nm excitation and 610 nm emission optimized for detection of GNCs. Live cell nuclear stain, Hoechst 33342, was used to define the location of nuclei.

The fluorescence images revealed a striking increase in GNC formation in cancerous cells as compared to non-cancerous cells after incubation with buffered chloroauric acid (Au³⁺) (FIG. 6A). Radiosensitization due to intracellular GNC formation (i.e., delivery of Au³⁺) was compared between cancerous and non-cancerous cells using a standard clonal assay. For radiosensitization, cells were incubated with 0.1 mM of buffered chloroauric acid in cell culture media for 24 hours. Then, the cells were lifted and plated in 30 mm culture dishes at optimized cell densities for observation of colony formation following irradiation with X-rays at dosages ranging from 0 to 8 Gy (XRAD SmART). The clonogenic assay showed a significantly greater radiosensitization effect from Au³⁺ treatment in cancer cells as compared to untreated control (FIG. 6B). Importantly, there was no significant radiosensitization in non-cancerous cells. The radiosensitization results correlated well with the fluorescence images showing greater production of GNCs by cancerous cells (FIG. 6A-6B). For LTPA pancreatic cancer cells the surviving fraction values decreased by a factor of 2.3x and 3.5x for radiation of 4 Gy and 6 Gy, respectively, in cells treated with gold atoms compared to untreated control (FIG. 6B). This relative decrease in surviving fraction is similar or better than previously reported values observed in cells treated with pre-synthetized GNPs. We believe that the observed improvement in radiation efficiency in killing cancer cells might be associated with a strong nuclear localization of in situ synthetized GNCs.

Summary of preliminary data

Taken together our preliminary data demonstrate that (i) intracellularly synthetized GNCs can produce a radiosensitization effect; (ii) in situ formation of GNCs is significantly greater in cancer pancreatic cells as compared to non-cancerous pancreatic cells; and (iii) there is substantial localization of the GNCs inside cell nuclei. These results provide a foundation for development of a novel paradigm-shifting radiosensitization strategy for clinically translatable RT of pancreatic tumors. Here we evaluate and optimize this strategy using a rigorous research plan that culminates with validation studies in clinically relevant models of pancreatic cancer.

Future work

Aim 1. Optimization and characterization of intracellular synthesis of GNCs and GNPs by cancer cells. 1.1. Optimize the dose of gold atoms and the timeframe for intracellular synthesis of GNCs. These studies will be carried out in a panel of human cancer pancreatic cell lines from more radiation resistant PANC-1 and BxPC3 cells to more sensitive HPAC, MIAPaCa-2 and AsPC-1 cells as well as patient derived pancreatic cancer cells. Non-cancer pancreatic cell line (HPDE) will be used as a normal control. In addition, we will evaluate in situ synthesis of GNCs in cells that are associated with a tumor microenvironment—murine (J774A.1, ATCC) and human (MV-4-11, ATCC) macrophages; murine (3T3, ATCC) and human (HUF, ATCC) fibroblasts.

In a typical experiment cells growing in sterile optical microplates will be treated with buffered chloroauric acid (HAuCl₄, Sigma-Aldrich) at concentrations ranging from 0.01 mM-10 mM for time periods up to 48 hours under standard cell incubation parameters (˜95% humidity, 5% CO2, 37° C., normal pH) in the cell media with 0, 5, 10, 15 and 20% FBS; note that phenol red-free media will be use as this indicator dye can interfere with optical measurements. All samples will be prepared at least in triplicate. Evaluation of GNC and GNP formation will be carried out every two hours with a BioTek Cytation 5 plate reader using fluorescence (561 nm excitation/610 nm emission) and UV-Vis absorbance acquired from the whole sample (i.e., cells+media) and the cells and the media alone; the samples will be staggered to allow long breaks between measurements. After media replacement, cell viability will be determined by an MTS assay; note the initial UV-Vis measurements from the cells alone will be used to correct for background absorbance at 490 nm. Then, cells and media from all samples will be analyzed for the total gold content by Inductively Coupled Plasma Mass Spectrometer (ICP-MS, Agilent). In a separate set of experiments longitudinal fluorescence and UV-Vis measurements will be carried out with non-cytotoxic doses of Au³⁺ (from the previous study) every hour up to the first 8 hours and then, at 20 h, 24 h, 28 h, 40 h, 44 h and 48h. Untreated cells will be used as controls and treatments with equimolar gold concentrations of albumin-GNCs and citrate-reduced 5 nm and PEGylated 5 nm spherical GNPs for comparison.

In these studies, fluorescence and UV-Vis data will provide kinetics of GNC/GNP bio-synthesis and changes in their concentration over time inside cells and in the surrounding media. These two methods are complimentary because fluorescence is sensitive to formation of very small GNCs and its intensity diminishes with transition to GNPs where UV-Vis has much better sensitivity due to a pronounced absorbance associated with plasmon resonances of the particles. ICP-MS quantifies the total gold content regardless of its physical state that will determine kinetics of gold uptake by various cells. These experiments will determine the optimum conditions (i.e., dose and time) for formation of intracellular GNCs and GNPs without cytotoxicity to normal cells. They will also identify parameters that provide the highest difference in formation of GNCs/GNPs between cancerous and normal cells. Final characterization of GNCs formation will be carried out by Flow Cytometry that will determine heterogeneity of GNC biosynthesis in different cell populations and will further quantify differences between normal and cancerous cells.

In addition, we will carry out initial evaluation of a potential role of cell- excreted vesicles, peptides, and nucleic acids in biosynthesis of GNCs. Cells will be grown for various periods of time (i.e., 12, 24 and 48 hours); care will be taken to make sure that the cells do not grow beyond confluence by adjusting the number of seeded cells. At each time point the optimum dose of Au³⁺ (from the studies above) will be applied to the cells and biosynthesis of GNCs/GNPs will be monitored using the methodology described above. Note that the cell media will not be replaced to preserve all biological substances released by the cells during growth. These experiments will determine if the cell conditioned media results in an extracellular formation of GNCs/GNPs and/or influences gold uptake by cells. Cells washed with a fresh media before addition of gold atoms will be used as controls.

1.2. Intracellular Distribution of GNCs as a Function of Time will be determine using confocal fluorescence (Leica TCS SP8 Confocal Microscope). In addition, samples at various time points that are associated with changes in fluorescence intensity and/or intracellular distribution of fluorescent GNCs will be analyzed by transmittance electron microscopy (TEM, JEM 1010, JEOL). The major goal of these studies is to understand spatiotemporal progression of GNC biosynthesis by cells. This knowledge will ultimately allow optimization of timing and parameters of RT with intracellularly synthetized GNCs. In a highly synergistic to this proposal study we are collaborating with Dr. S. H. Cho in the Department of Radiation Physics at the M.D. Anderson Cancer Center on development and validation of Monte Carlo computational modeling of gold-mediated radiosensitization that can predict radiation dose enhancement based on distribution of GNCs/GNPs.^{16,35,36} Further, a number of reported studies proposed various mechanisms of in situ biosynthesis of GNCs/GNPs, including the potential role of various cellular compartments that are rich in biomolecules with sufficient reducing potential for gold atoms reduction including (i) the cytoplasmic cell membrane that contains reducing enzymes and glycosylated moieties;⁶ (ii) reactive oxygen species (ROS), glutathione (GSH) and glutathione disulfide (GSH-GSSG), nicotinamide adenosine dinucleotide phosphate hydrogenase enzyme (NAD(P)H) and QOH-1 enzymes in the cytoplasm;^{8,14} (iii) and nucleotides in the nucleus.⁴⁷ Our studies can provide an insight into which compartments and in what sequence are involved in synthesis and trafficking of GNCs.

In a typical experiment, cells will be grown in a live cell imaging chamber under normal conditions (˜95% humidity, 5% CO2, 37° C., and normal pH) in a cell culture media. Confocal fluorescence images will be collected before and in 20 minute intervals after administration of gold atoms up to a 24 hour period. Initial cell localization will be determined using bright-field imaging. Cellular nuclei will be stained with the Hoechst stain (live cells nuclear stain). Cytoplasmic cell membranes will be labeled with DiO membrane tracer (484 nm excitation/501 nm emission, ThermoFisher) that does not overlap with fluorescence of GNCs based on our preliminary data; other lipophilic carbocyanine tracers can be explored if needed, e.g., DiR (750 nm exc./780 nm em.).

After this longitudinal study, in a separate set of experiments we will collect samples for TEM analyses to determine cellular distribution of GNCs in the context of cellular compartments and organelles with higher resolution. In addition, the total amount of gold in the cytoplasm (plus cytoplasmic membrane) and the nucleus will be determined using ICP-MS. To this end we will separate nuclei using mechanical lysis (Tip sonication, QSonica) followed by differential centrifugation (LK-90 Ultracentrifuge) as described in detail by Lodish and coworkers⁴⁸. Image analysis will be done in ImageJ or IMARIS (Bitplane). Cell and nucleus boundaries will be segmented using fluorescence of cytoplasmic membrane labeled with DiO and Hoechst stain, respectively. Then intensity of GNC fluorescence inside and outside cells as well as in cytoplasm versus nuclei will be quantified. Statistical analyses. Student's t test will be used to compare Gaussian-distributed data, whereas the nonparametric Mann-Whitney test (two-group comparison) will be used to analyze non-normally distributed data. P values of less than 0.05 will be considered statistically significant.

Expected outcomes: Significantly higher efficiency of GNC biosynthesis by pancreatic cancer cells relative to pancreatic normal cells. Highly efficient uptake of gold atoms from the surrounding media by cancer cells with substantial accumulation of GNCs inside cell nuclei. Optimized dose of gold atoms that results in efficient synthesis of GNCs by cancer cells with minimum cytotoxicity to normal pancreatic cells.

Possible obstacles: If we discover a high fraction of GNP formation inside cells, two-photon luminescence can be used for their detection, since GNPs do not exhibit sufficiently high one-photon fluorescence cross section as opposed to GNCs. Multiple dosing of gold atoms can be implemented if a single dose exhibits substantial cytotoxicity towards normal cells.

Aim 2: Evaluate radiosensitization efficacy of in situ synthetized GNCs in models of pancreatic cancer.

2.1. Compare RT of cancer and normal cells after treatment with gold atoms in vitro. A panel of normal and pancreatic human cells described above will be treated under optimum conditions (from Aim 1) with gold atoms. Then the ability of biosynthesized GNCs/GNPs will be evaluated in a clonogenic survival assay after irradiation with 0, 2, 4, 6, 8 or 10 Gy of radiation. The cell survival will be monitored during the standard period of ˜10-16 days.⁴⁹ The data will be fit to the linear-quadratic model of cellular response to radiation and parameters such as surviving fraction at 2 Gy (SF2), α/β ratio, and D₀ will be used to compare treatment efficiencies. These data will be compared with fluorescence microscopy, TEM and ICP-MS results (Aim 1) to correlate treatment response with cellular uptake, internalization, and intracellular distribution. Treatments with equimolar gold concentrations of albumin-GNCs and 5 nm spherical GNPs will be used for comparison.

Evaluation of mechanism of radiosensitization in vitro. Gamma H2AX foci (markers of unrepaired DNA strand breaks) will be quantified at multiple time points following radiation in the absence and presence of gold atom treatment. Activation of DNA repair pathways (ATM, ATR, Ku70, Ku80, DNAPKcs, chk1 and chk2 Western blot analyses), mitochondrial oxidative stress pathways (membrane potential JC-1 assay and NADP/NADPH ratio), and cell membrane lipid peroxidation (TBARS assay) will be evaluated in treated cells. Blocking studies with N-acetyl cysteine will determine whether oxidative stress induced effects are reversible.

The JC-1 assay measures the charge potential of the mitochondria of cells through the fluorometric ratio of the JC-1 dye (ThermoFisher Scientific, Waltham, Mass.). JC-1 is a cationic carbocyanine dye that accumulates in mitochondria. The dye exists as a monomer at low concentrations and yields green fluorescence, similar to fluorescein. At higher concentrations, the dye forms J-aggregates that exhibit a broad excitation spectrum and an emission maximum at ˜590 nm. These characteristics make JC-1 a sensitive marker for mitochondrial membrane potential. In other words, mitochondrial depolarization is indicated by a decrease in the red/green fluorescence intensity ratio. Mitochondrial depolarization is an indicator of reduced cell viability.

Expected outcomes: Although not comprehensive, these studies will identify the magnitude of and mechanisms of radiosensitization of cancer cells by GNCs/GNPs generated intracellularly via applications of ionic gold.

Possible obstacles: While the emphasis of the mechanistic studies is on DNA damage, the parallel investigation of mitochondrial and cell membrane signaling alterations after radiation will allow identification of non-DNA adaptive responses of cells to radiation.

2.2. Toxicity study will be performed in C57BL6 mice without tumors. Eight mice per group (4 male and 4 female) will be evaluated for toxicity of 2 administration routes (i.v. and i.p.) at 3 dose levels and at 2 time points (1 week and 4 weeks). Toxicity assessment will include mouse weight, biochemistry panel (renal function, liver function tests, and electrolytes), hematology panel and histopathological evaluation of normal organs (liver, spleen, heart, lung, pancreas, and kidney) as described previously by us⁵⁰.

2.3. In vivo biodistribution, cellular internalization, and subcellular trafficking will be determined in murine models of pancreatic cancer. Ionic gold will be injected at 3 doses into pancreatic tumors under ultrasound (US) guidance. If US is unable to provide sufficient contrast, the mouse abdomen will be opened under anesthesia and the treatment will be made under visual guidance. Eight animals (4 male and 4 female) will be sacrificed at 3 time points based on in vitro cell studies (Aim 1); in addition, we will monitor formation of GNCs using IVIS Spectrum system (Caliper). Distribution of in situ synthetized GNCs/GNPs in the pancreas, including uptake by cancer cells, will be determined in slices of excised tumors using a combination of confocal fluorescent microscopy, MALDI (Waters Synapt G2-Si), and histology with silver stain. These experiments will be used to fine tune the dose of gold atoms and timing of RT for in vivo radiosensitization study.

2.4. Evaluation of radiosensitization in vivo. A tumor regrowth delay experiment will be used to determine radiosensitization in vivo. An orthotopic human pancreatic tumor model will be used in these experiments. This model closely reproduces the complex biology of pancreatic human cancer.^{51,52} Once tumors are a few mm in diameter (as determined by US imaging), they will be given intratumoral injections of gold atoms in saline (at the optimum dose from Aims 2.1 and 2.3). A pre-made nanomaterial (Albumin-GNC or 5 nm spherical GNPs) with the best radiosensitization (Aim 2.1) will be used for comparison. Radiotherapy will be administered after a time delay determined in the Aim 2.3 and confirmed by IVIS fluorescence, to allow diffusion of gold atoms throughout the tumor, intracellular nanoparticle reduction, and nuclear localization. A customized collimator will be used to administer a dose of 10 Gy using a small animal irradiator (XRAD255). Tumor size by US and mouse weight will be measured three times a week and mice will be euthanized when they experience a 20% weight loss from baseline. Tumor volume measurements (based of US) will be used to determine the time to tumor volume doubling in each treatment group (control, radiation, GNPs (or albumin-GNC), ionic gold, GNPs + radiation, and ionic gold + radiation).

Statistical analyses. The primary comparison will be between (i) radiation alone and (ii) radiation + ionic gold. We will use t test or Mann-Whitney test for two-group comparisons and ANOVA or Krustal-Wallis test for multiple-group comparisons. For the repeated measures (e.g., tumor size), we will use the linear mixed model. Subgroup analysis will be conducted for male and female mice. The sample size chosen for this experiment is based on estimates of a mean delay time of ˜7 days [standard deviation (SD) of ˜3 days] for the control (radiation alone) group of tumors to double in volume. To detect a mean difference of 10 days for the test group [90% power, two-sided a of 5%], we will need 8 animals per group assuming similar SDs in the test group. If it turns out that the SD in the control group is considerably different from that in the test group (say 3 days vs. 6 days), then we would need 10 animals per group. We will use equal numbers of male and female animals.

Expected outcomes: These studies will evaluate the radiosensitization of pancreatic tumors by intracellular GNC/GNP formation in vivo.

Possible obstacles: Injection of ionic gold can also be monitored through photoacoustic imaging that can provide higher needle and tumor contrast. In case US is not sensitive enough to monitor tumor growth, we will switch to a 7T MRI (Bruker).

Conclusion

Our compelling preliminary data in pancreatic cells provide the scientific premise of our strategy wherein gold atoms are used for radiosensitization of PDAC that is based on the following observations: (i) gold atoms have diffusion kinetics similar to other soluble salts and thus can penetrate throughout the tumor more readily than molecules or nanoparticles;³⁷⁻⁴⁰ (ii) intracellular GNP formation preferentially occurs via interactions with cancerous cells;^{6,8,9,11,12} (iii) biosynthesized GNPs innately localize within the nucleus;⁵⁻⁷ and (iv) GNPs have a high radiosensitization efficiency if located within the nucleus of target cells.^{28, 41-43} We see it as a highly innovative and exciting opportunity to greatly improve radiosensitization efficiency of cancer cells in situ.

Example 2

We followed up on the experiments described in Example 1, as follows:

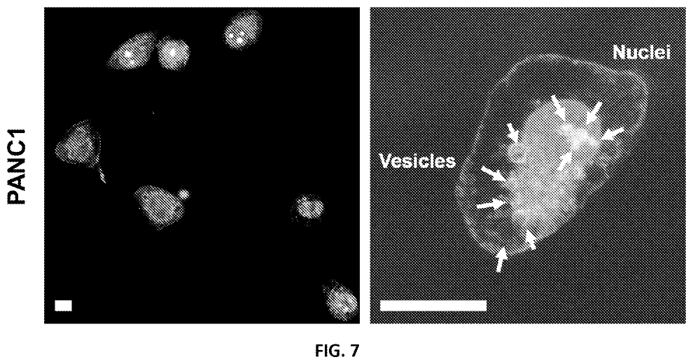

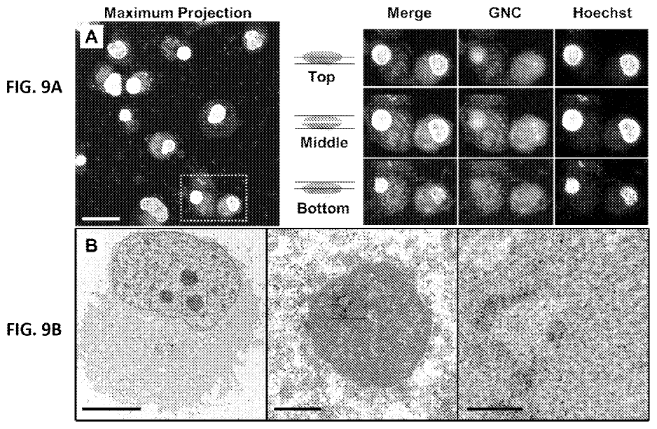

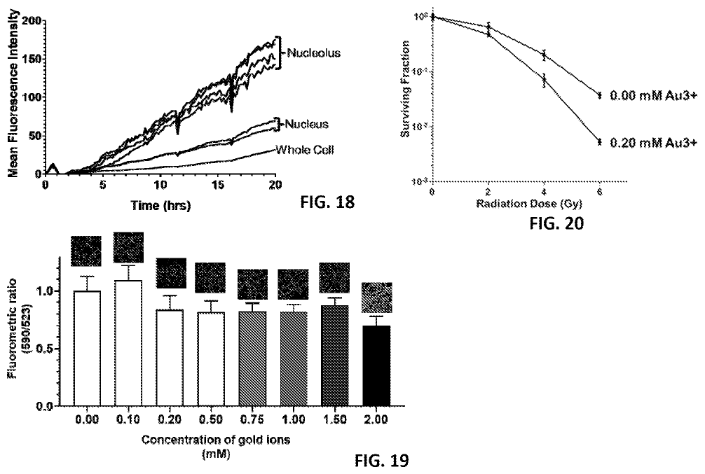

2.1. Intracellular distribution and time dependence of gold nanocluster (GNC) in situ formation. We used a combination of TEM (FIG. 9B) and confocal fluorescence microscopy (FIG. 7) to demonstrate a high level of intranuclear localization of intracellularly formed GNCs. Then, we used longitudinal live cell confocal fluorescence imaging to observe time dependence of the intracellular distribution of GNCs formed through biomineralization of Au³⁺. We found that biosynthesis of GNCs occurred simultaneously throughout the cells, however, at different rates in different subcellular regions. The nucleolus had the highest rate of GNC formation (FIG. 18). This observation indicates that GNC biosynthesis occurred without additional intracellular transport and that Au³⁺ ions were able to permeate throughout the cell.

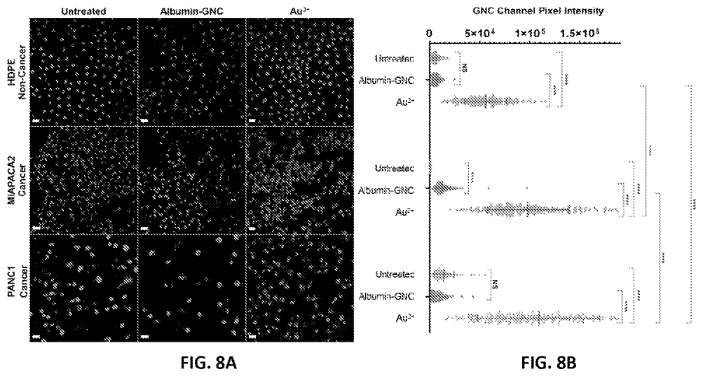

2.2. Comparisons of efficiency of the GNC in situ synthesis by cancer and normal cells. We compared GNC biosynthesis between normal (HPDE) and cancerous (Mia-PaCa-2 and PANC1) pancreatic cells using fluorescent confocal microscopy (FIG. 8A). In addition, we compared the efficiency of the intracellular synthesis with extracellular uptake of prefabricated albumin coated GNCs (FIG. 8A). We showed that cancerous pancreatic cells exhibited ˜2-fold greater fluorescence due to GNC biomineralization than non-cancerous HPDE (FIG. 8B). Further, there was 12.20- and 7.69-times greater fluorescence due to in situ synthesis relative to cellular uptake of prefabricated GNCs in PANC1 and Mia-PaCa-2 cells, respectively (FIG. 8B).

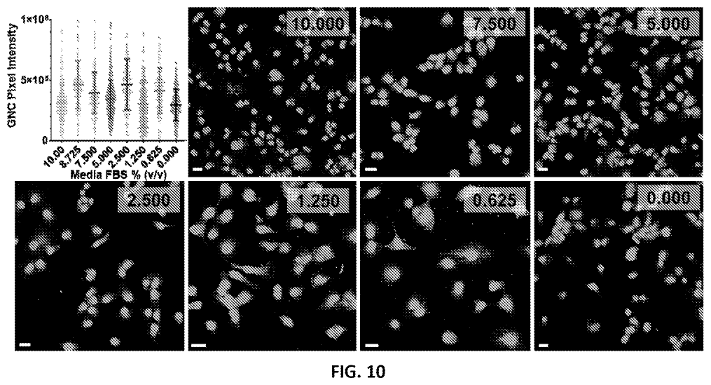

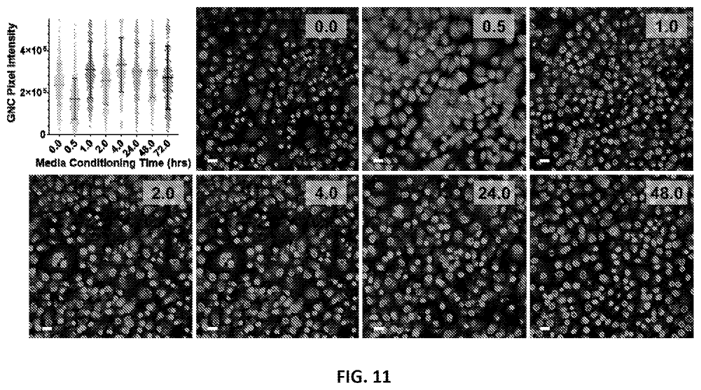

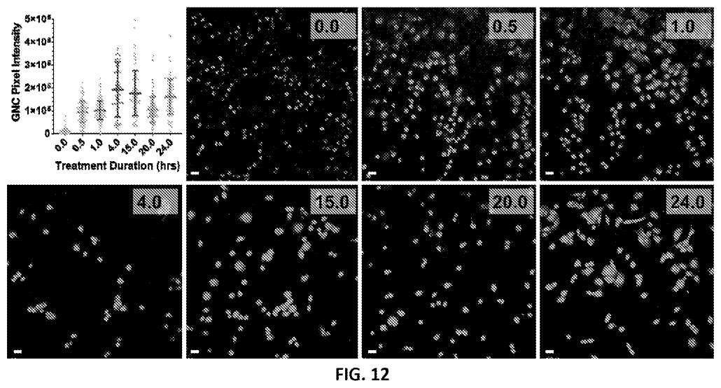

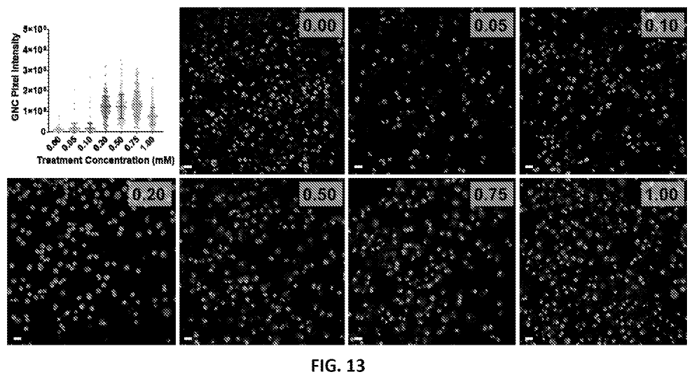

2.3. Optimization of conditions for intracellular synthesis of GNCs. We determined optimal conditions for intracellular synthesis of fluorescent GNCs by PANC1 cells through variations in (1) how long cells condition media before addition of Au³⁺ ions (FIG. 11), (2) the concentration of FBS within the media (FIG. 10), (3) concentration of added Au³⁺ ions (FIGS. 13), and (4) incubation time (FIG. 12). We used the following standard conditions: 24 hr cell conditioning time, 10% FBS, 1.00 mM Au³⁺, and 24 hr Au³⁺ treatment duration. In the optimization studies one of these parameters was varied at a time as shown in FIG. 10-FIG. 13. No significant changes were observed due to variations either in duration of media conditioning by cells up to 72-hr or changes in FBS concentration between 0-10% (v/v) in the media. We observed significant formation of GNCs at Au³⁺ concentrations between 0.20-0.75 mM, with fluorescence reduced at concentrations greater than 1.00 mM, possibly, due to formation of larger non-fluorescent gold nanoparticles (GNPs). We observed fluorescence as early as 30 minutes after addition of gold atoms with a significant increase up to 4 hours.

2.4. Characterize cytotoxicity of gold atom treatment. An MTS viability assay was used with 0-2.0 mM Au³⁺ dose range. There were no impacts on viability to PANC1 cells for concentrations 0.0-0.20 mM Au³⁺, with a decrease in cell viability to ˜81% at 0.75 mM. MTS assay was not usable at concentrations >0.75 mM Au³⁺ due to intracellular formation of larger GNPs and their absorbance interfering with the MTS results. Therefore, at higher Au³⁺ we switched to AO/PI live-dead staining (FIG. 15) and JC-1 mitochondrial depolarization assays (FIG. 19). JC-1 assay indicated cell viability ˜80% for concentrations between 0.20-1.50 mM Au³⁺.

2.5. Evaluate radiosensitization efficacy and radiosensitization mechanisms for in situ synthetized GNCs. Based on the studies described in Sections 2.3-2.4, we selected the following conditions for radiosensitization studies in cell culture: 24 hr media conditioning by cells, 10% FBS (v/v) within, and 0.20 mM Au³⁺ for 24 hrs.

A standard clonogenic assay was used to evaluate radiosensitization efficacy of intracellular synthetized GNC in PANC1 cells (FIG. 20). Intracellular formed GNCs resulted in a substantial radiosensitization of PANC1 cells as was measured by differences between cells pretreated with Au³⁺ and untreated control in surviving fraction at radiation dosages of 2, 4, and 6 Gy with average respective surviving fractions of 64.9, 20.3, and 3.8% without ionic gold vs. 47.3, 7.3, and 2.2% with ionic gold (p<0.0005) (FIG. 20). Dose enhancement factor at 10% surviving fraction (DEF10%) was calculated at 1.317 indicative of a strong radiosensitization in Au³⁺ treated cells.

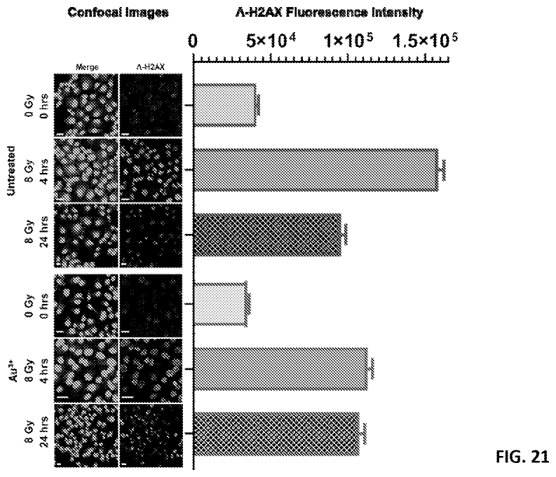

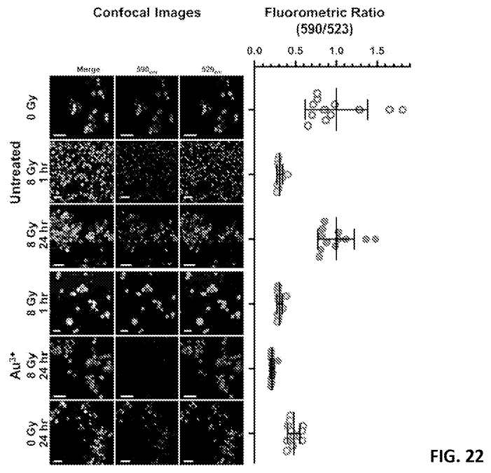

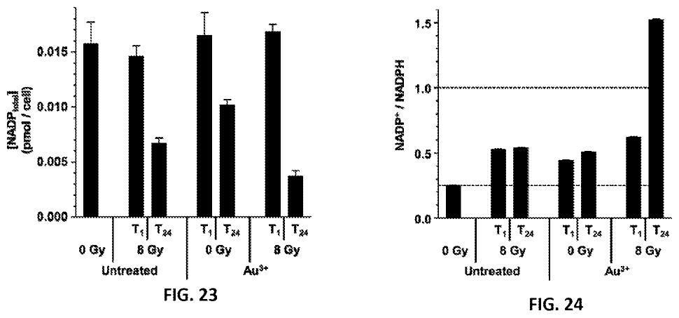

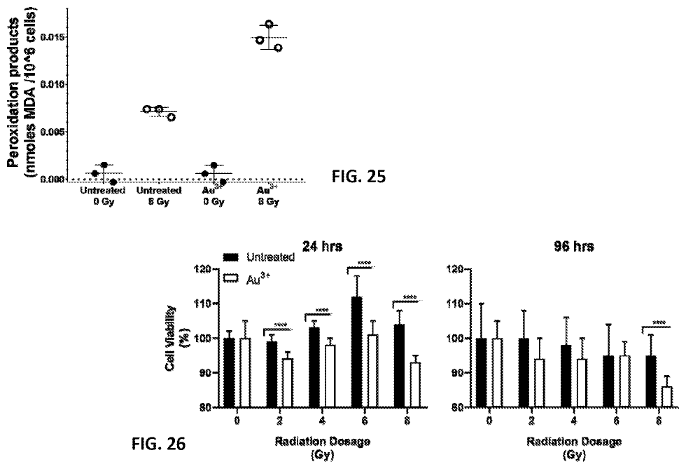

Further, we characterized the mechanisms of radiosensitization through (i) quantification of double stranded DNA breaks via y-H2AX Foci (FIG. 21), (ii) mitochondrial polarization via JC-1 assay (FIG. 22), (iii) NADP total (FIG. 23) and NADP:NADPH ratio (FIG. 24) via NADP(H) assay, and (iv) peroxidation production via TBARS assay (FIG. 25). In a short-term study of double stranded DNA breaks 4 hrs after 8 Gy irradiation, we surprisingly found 29% fewer DNA breaks in the Au³⁺ treated cells than in untreated control. However, the DNA breaks recovered to baseline at 24 hr. post-irradiation in the control cells, while there was no significant repair in the treated cells. Similarly, there was no recovery in the mitochondrial depolarization of pancreatic cancer cells pretreated with Au³⁺ ions while the mitochondria return to normal polarization state for control cells at 24 hr post-irradiation (FIG. 22). NADP(H) assay showed that at 24 hr. post-irradiation the NADP total was ˜43% lower (FIG. 23) and the NADP:NADPH ratio was 181% larger (FIG. 24) for Au³⁺ treated cells than for the non-treated cells, respectively, that indicates significant dysregulation of cellular metabolism in radiosensitized cells. For TBARS assay showed ˜2x increase in peroxidation product formation in the Au³⁺ treated cells at 24 hr. post irradiation (FIG. 25).

Specific Objectives of our studies were to: (1) determine intracellular distribution and kinetics of intracellular synthesis of GNCs; (2) determine and optimize environmental factors that impact intracellular synthesis of GNCs; (3) compare efficiency of the GNC synthesis by normal and cancer cells; (4) compare efficiency of intracellular synthesis of GNCs with cellular uptake of prefabricated GNCs; (5) characterize cytotoxicity of cell treatment with Au³⁺ ions; (6) evaluate radiosensitization efficacy of pancreatic cancer cells in cell culture; (7) determine the underlying mechanism of the radiosensitization effect.

Significant Results were tightly connected to the Specific Objectives. Specifically, we (i) observed high colocalization of intracellular GNCs in nucleolus; (ii) determined that intracellular GNC synthesis occurs at higher efficiency in cancerous compared to normal pancreatic cells; (iii) showed that intracellular GNC synthesis is more efficient for gold internalization than uptake of prefabricated GNCs; (iv) optimized conditions for cell treatment with Au³⁺ ions; (v) demonstrated efficient radiosensitization of pancreatic cancer cells; (vi) showed that radiosensitization leads to effective suppression of cell repair mechanisms post X-ray irradiation.

These studies prove our key hypothesis that small gold atoms can yield GNCs after specific uptake by cancer cells that results in cancer cell radiosensitization to radiotherapy. Further, normal pancreatic cells do not substantially produce GNCs.

In addition to the figures referred to above, other figures presented herein relate to Example 2.

FIG. 9A shows fluorescence images of gold nanoclusters formed resulting from 24 hr. treatments of 1.00 mM Au³⁺ (as chloroauric acid) in full cell media to PANC1 pancreatic cancer cells with Hoechst nuclear stain. The cross-sectional imaging demonstrates the gold nanocluster fluorescence is internal to the cell nuclei. Cells are live during imaging. Scale bars are 20 μm.

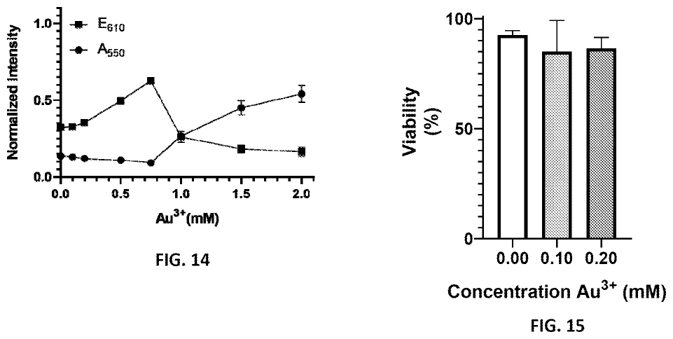

FIG. 14 graphs fluorescent nanoparticle formation (ex560/em610 nm) with plasmonic nanoparticle formation (A550 nm) as a function of Au³⁺ treatment concentration made over 24 hours in full cell media to PANC1 pancreatic cancer.

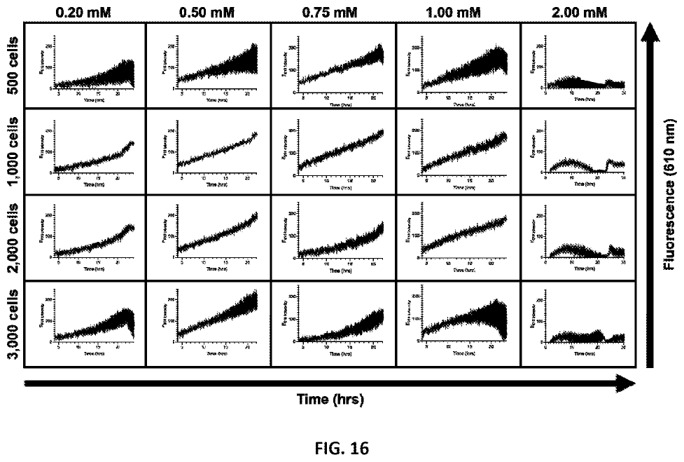

FIG. 16 shows fluorescent nanoparticle formation (emission at 610 nm) as a function of Au³⁺treatment concentration and cell density made over a 20 hour period in full cell media to PANC1 pancreatic cancer.

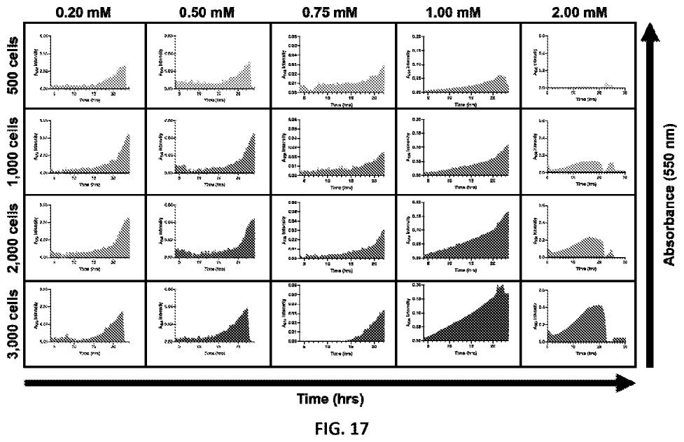

FIG. 17 shows plasmonic nanoparticle formation (A550 nm) as a function of Au³⁺ treatment concentration and cell density made over a 20 hour period in full cell media to PANC1 pancreatic cancer.

FIG. 18 shows longitudinal Pancl pancreatic cancer cell fluorescence across a 20 hr time period resulting from 0.20 mM treatment of Au³⁺ (as chloroauric acid) in full cell media. Generally, GNC formation was greatest in the nucleolus, with lesser amounts in the nucleus and even lesser amounts in the cell outside of the nucleus.

FIG. 26 presents evidence of radiosensitization, quantifying the cell viability resulting from X-ray damage through MTT assay measured 24 and 96 hours after x-ray irradiation, resulting from 24 hour, 0.20 mM Au³⁺ treatments (right bar in each dosage pair) compared against non-treated (left bar in each dosage pair) combined with either 0 Gy or 8 Gy x-ray irradiation. Treatments are in full cell media to PANC1 pancreatic cancer. As can be seen, after 24 hours, the treatment resulted in significantly lower cell viability than the untreated controls at all radiation dosages greater than 0 Gy.

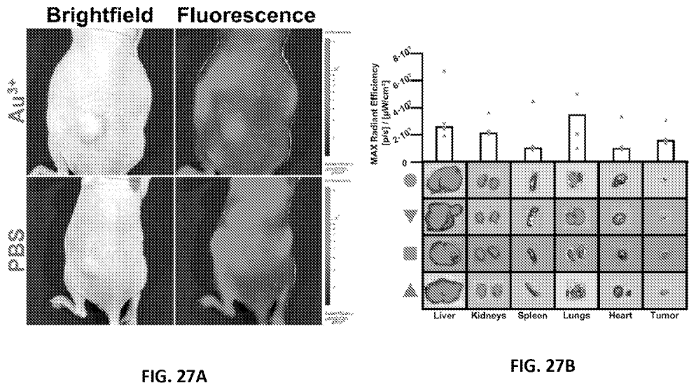

FIG. 27A. Fluorescence nanoparticle formation through IVIS imaging (ex610/em660 nm) of nanoparticle formation in PANC1 xenografts in nu/nu mice 48 hours after treatment with 1.00 mM Au³⁺ (as chloroauric acid).

FIG. 27B. Fluorescence of extracted organs of treated mice shown in FIG. 27A.

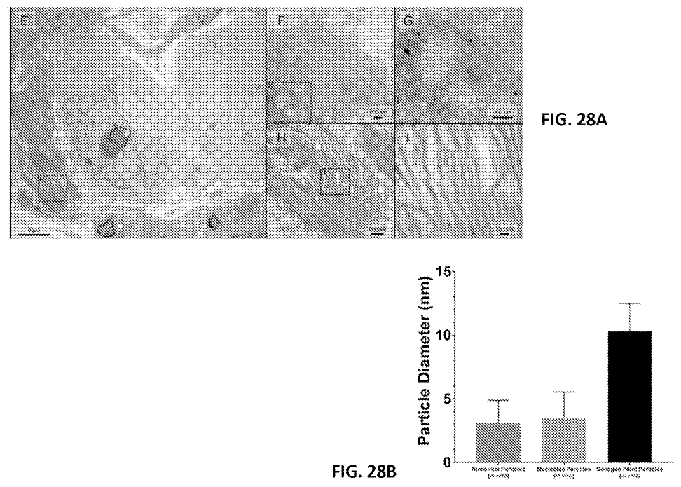

FIG. 28A shows transmission electron micrographs of nanoparticle formation in PANC1 xenografts in nu/nu mice 48 hours after treatment with 1.00 mM Au³⁺ (as chloroauric acid).

FIG. 28B quantifies particle diameters from the transmission electron micrographs shown in FIG. 28A.

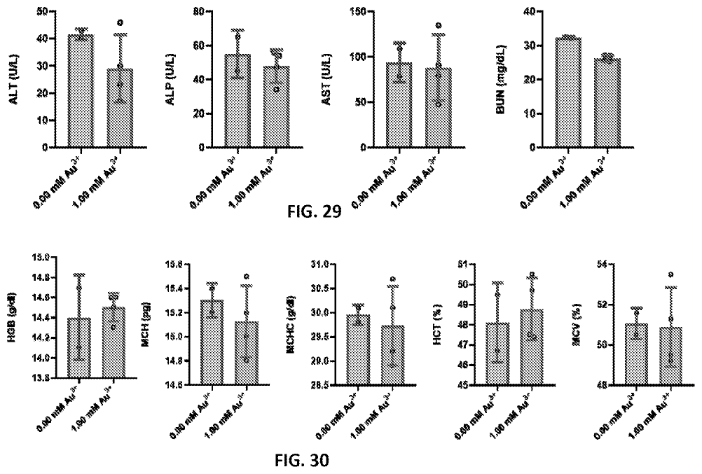

FIG. 29 shows blood chemistry and hematology data following nanoparticle formation in PANC1 xenografts in nu/nu mice 48 hours after treatment with 1.00 mM Au³⁺ (as chloroauric acid) vs. controls.

FIG. 30 shows blood chemistry and hematology data following nanoparticle formation in PANC1 xenografts in nu/nu mice 48 hours after treatment with 1.00 mM Au³⁺ (as chloroauric acid) vs. controls.

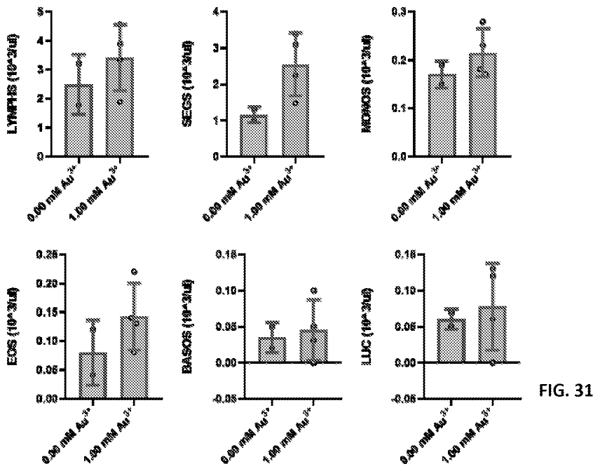

FIG. 31 shows blood chemistry and hematology data following nanoparticle formation in PANC1 xenografts in nu/nu mice 48 hours after treatment with 1.00 mM Au³⁺ (as chloroauric acid) vs. controls.

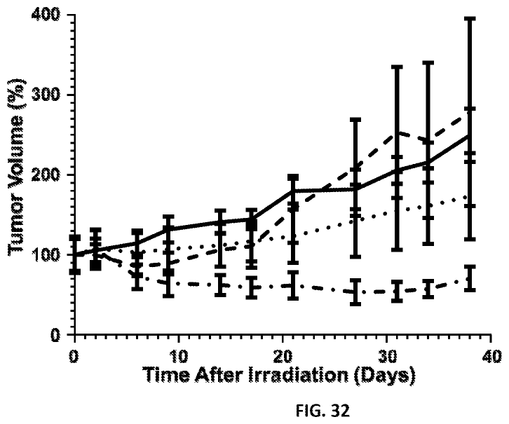

FIG. 32 shows evidence of radiosensitization effect following nanoparticle formation in PANC1 xenografts in nu/nu mice 48 hours after treatment with 1.00 mM Au³⁺ (as chloroauric acid) (bottom and uppermost traces) compared to non-treated (middle two traces) by tumor volume measurements occurring after 10 Gy X-ray irradiation.

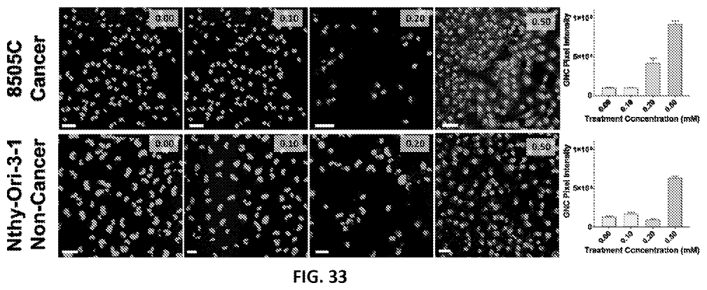

FIG. 33 shows fluorescence images of gold nanoclusters formed resulting from 24 hr. treatments of Au³⁺ (as chloroauric acid) in full cell media to 8505C thyroid cancer cells and Nthy-Ori-3-1 normal thyroid cells with Hoechst nuclear stain (blue), under varied treatment Au³⁺ treatment concentrations. Cells are live during imaging. Scale bars are 20 μm.

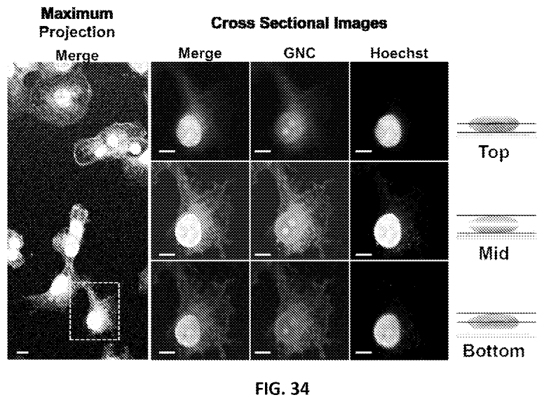

FIG. 34 shows fluorescence images of gold nanoclusters formed resulting from 24 hr. treatments of 1.00 mM Au³⁺ (as chloroauric acid) in full cell media to 8505C thyroid cancer cells with Hoechst nuclear stain. Cross sectional imaging demonstrates the gold nanocluster fluorescence is internal to the cell nuclei. Cells are live during imaging. Scale bars are 20 μm.

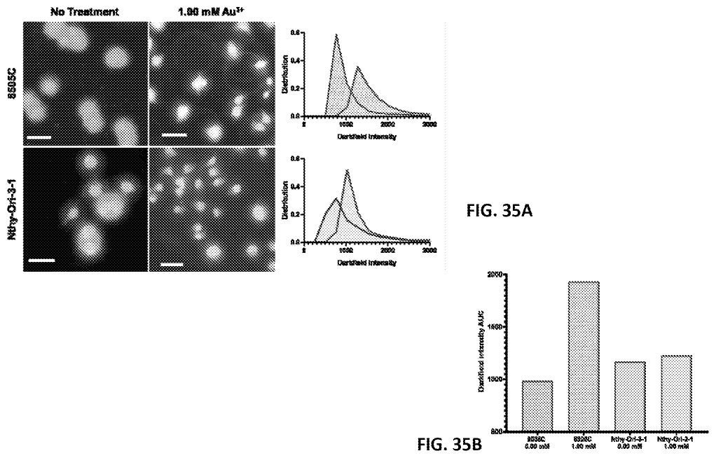

FIG. 35A. Darkfield images of gold nanoparticle formation resulting from 24 hr. treatments of 1.00 mM Au³⁺ (as chloroauric acid) in full cell media to 8505C thyroid cancer and Nthy-Ori-3-1 normal thyroid cells with Hoechst nuclear stain. Cells are fixed for imaging. Scale bars are 20 μm.

FIG. 35B. Darkfield intensity areas under the curve (AUCs) for the images shown in FIG. 35A.

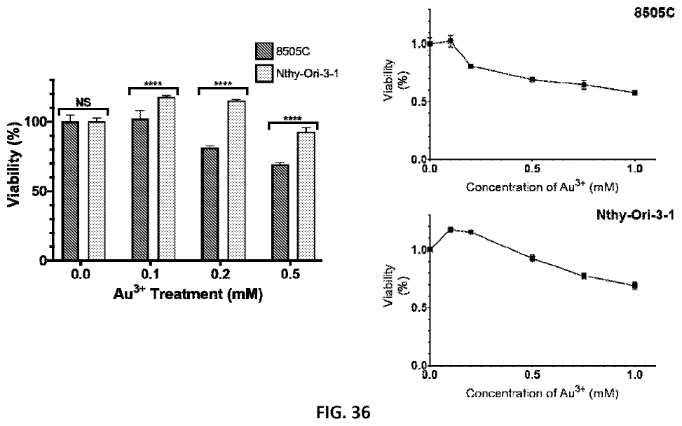

FIG. 36 shows cell viability as a function of 24 hour Au³⁺ treatments at varied concentrations determined via MTT assay and in full cell media to 8505C thyroid cancer and Nthy-Ori-3-1 normal thyroid cells. Au³⁺ treatment gave a significantly lower viability of cancer cells than normal cells at all concentrations.

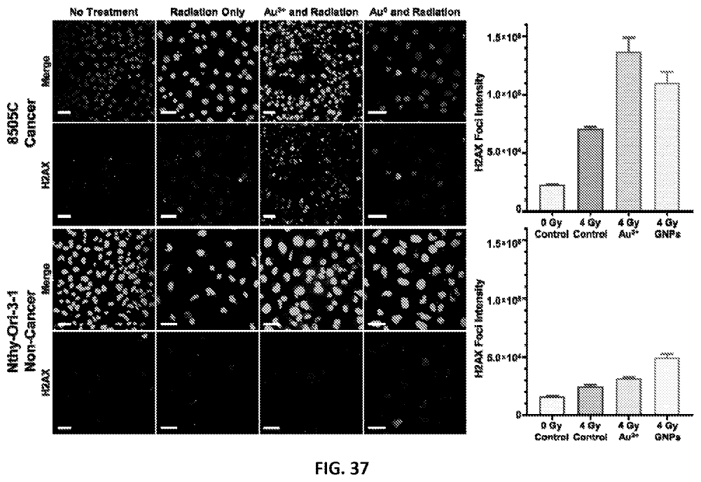

FIG. 37 shows evidence of radiosensitization via induced double stranded DNA breaks in thyroid cancer quantifying gamma H2AX foci through fluorescent antibody staining measured at 24 hours after x-ray irradiation, resulting from 24 hour treatments of 0.20 mM of either Au³⁺ or Au⁰ prefabricated gold particles (GNPs) compared against non-treated combined with either 0 Gy or 8 Gy x-ray irradiation. Treatments are in full cell media.

REFERENCES

1. Jain, R.K. Delivery of molecular and cellular medicine to solid tumors. J Control Release 53, 49-67 (1998).

2. Huang, X.H., Jain, P.K., El-Sayed, I.H. & El-Sayed, M.A. Plasmonic photothermal therapy (PPTT) using gold nanoparticles. Lasers in Medical Science 23, 217-228 (2008).

3. Jain, R.K. & Stylianopoulos, T. Delivering nanomedicine to solid tumors. Nat Rev Clin Oncol 7, 653-664 (2010).

4. Wong, C., Stylianopoulos, T., Cui, J., Martin, J., Chauhan, V.P., Jiang, W., Popovic, Z., Jain, R.K., Bawendi, M.G. & Fukumura, D. Multistage nanoparticle delivery system for deep penetration into tumor tissue. Proc Natl Acad Sci USA 108, 2426-2431 (2011).

5. Anshup, Venkataraman, J.S., Subramaniam, C., Kumar, R.R., Priya, S., Kumar, T.S., Omkumar, R., John, A. & Pradeep, T. Growth of gold nanoparticles in human cells. Langmuir 21, 11562-11567 (2005).

6. Shamsaie, A., Jonczyk, M., Sturgis, J.D., Robinson, J.P. & Irudayaraj, J. Intracellularly grown gold nanoparticles as potential surface-enhanced Raman scattering probes. Journal of biomedical optics 12, 020502 (2007).

7. Liu, Z., Hu, C., Li, S., Zhang, W. & Guo, Z. Rapid intracellular growth of gold nanostructures assisted by functionalized graphene oxide and its application for surface-enhanced Raman spectroscopy. Analytical chemistry 84, 10338-10344 (2012).

8. El-Said, W.A., Cho, H.Y., Yea, C.H. & Choi, J.W. Synthesis of metal nanoparticles inside living human cells based on the intracellular formation process. Advanced materials 26, 910-918 (2014).

9. West, A.L., Schaeublin, N.M., Griep, M.H., Maurer-Gardner, E.I., Cole, D.P., Fakner, A.M., Hussain, S.M. & Kama, S.P. In situ synthesis of fluorescent gold nanoclusters by nontumorigenic microglial cells. ACS applied materials & interfaces 8, 21221-21227 (2016).

10. Zhao, C., Du, T., Rehman, F.U., Lai, L., Liu, X., Jiang, X., Li, X., Chen, Y., Zhang, H. & Sun, Y. Biosynthesized gold nanoclusters and iron complexes as scaffolds for multimodal cancer bioimaging. Small 12, 6255-6265 (2016).

11. Drescher, D., Traub, H., Buchner, T., Jakubowski, N. & Kneipp, J. Properties of in situ generated gold nanoparticles in the cellular context. Nanoscale 9, 11647-11656 (2017).

12. Singh, A.V., Jahnke, T., Kishore, V., Park, B.-W., Batuwangala, M., Bill, J. & Sitti, M. Cancer cells biomineralize ionic gold into nanoparticles-microplates via secreting defense proteins with specific gold-binding peptides. Acta biomaterialia 71, 61-71 (2018).

13. Rehman, F.U., Du, T., Shaikh, S., Jiang, X., Chen, Y., Li, X., Yi, H., Hui, J., Chen, B. & Selke, M. Nano in nano: Biosynthesized gold and iron nanoclusters cargo neoplastic exosomes for cancer status biomarking. Nanomedicine: Nanotechnology, Biology and Medicine 14, 2619-2631 (2018).

14. Wang, J., Zhang, G., Li, Q., Jiang, H., Liu, C., Amatore, C. & Wang, X. In vivo self-bio-imaging of tumors through in situ biosynthesized fluorescent gold nanoclusters. Scientific reports 3, 1157 (2013).

15. Forestier, J. Rheumatoid arthritis and its treatment by gold ions. The lancet 224, 646-648 (1934).

16. Krishnan, S., Chadha, A.S., Suh, Y., Chen, H.C., Rao, A., Das, P., Minsky, B.D., Mahmood, U., Delclos, M.E., Sawakuchi, G.O., Beddar, S., Katz, M.H., Fleming, J.B., Javle, M.M., Varadhachary, G.R., Wolff, R.A. & Crane, C.H. Focal Radiation Therapy Dose Escalation Improves Overall Survival in Locally Advanced Pancreatic Cancer Patients Receiving Induction Chemotherapy and Consolidative Chemoradiation. Int J Radiat Oncol Biol Phys 94, 755-765 (2016).

17. Jemal, A., Siegel, R., Ward, E., Murray, T., Xu, J., Smigal, C. & Thun, M.J. Cancer statistics, 2006. CA Cancer J Clin 56, 106-130 (2006).

18. Sener, S.F., Fremgen, A., Menck, H.R. & Winchester, D.P. Pancreatic cancer: a report of treatment and survival trends for 100,313 patients diagnosed from 1985-1995, using the National Cancer Database. J Am Coll Surg 189, 1-7 (1999).

19. Cairns, R., Papandreou, I. & Denko, N. Overcoming physiologic barriers to cancer treatment by molecularly targeting the tumor microenvironment. Mol Cancer Res 4, 61-70 (2006).

20. Cai, W. & Chen, X. Anti-angiogenic cancer therapy based on integrin alphavbeta3 antagonism. Anti-cancer agents in medicinal chemistry 6, 407-428 (2006).

21. Huang, Y.F., Chang, H.T. & Tan, W. Cancer cell targeting using multiple aptamers conjugated on nanorods. Anal Chem 80, 567-572 (2008).

22. Janssen, M.L., Oyen, W.J., Dijkgraaf, I., Massuger, L.F., Frielink, C., Edwards, D.S., Rajopadhye, M., Boonstra, H., Corstens, F.H. & Boerman, O.C. Tumor targeting with radiolabeled alpha(v)beta(3) integrin binding peptides in a nude mouse model. Cancer research 62, 6146-6151 (2002).

23. Lowery, A.R., Gobin, A.M., Day, E.S., Halas, N.J. & West, J.L. Immunonanoshells for targeted photothermal ablation of tumor cells. International journal of nanomedicine 1, 149-154 (2006).

24. Xie, H., Diagaradjane, P., Deorukhkar, A.A., Goins, B., Bao, A., Phillips, W.T., Wang, Z., Schwartz, J. & Krishnan, S. Integrin alphavbeta3-targeted gold nanoshells augment tumor vasculature-specific imaging and therapy. International journal of nanomedicine 6, 259-269 (2011).

25. Huguet, F., Goodman, K.A., Azria, D., Racadot, S. & Abrams, R.A. Radiotherapy technical considerations in the management of locally advanced pancreatic cancer: American-French consensus recommendations. Int J Radiat Oncol Biol Phys 83, 1355-1364 (2012).

26. Herman, J.M., Chang, D.T., Goodman, K.A., Dholakia, A.S., Raman, S.P., Hacker-Prietz, A., Iacobuzio-Donahue, C.A., Griffith, M.E., Pawlik, T.M., Pai, J.S., O'Reilly, E., Fisher, G.A., Wild, A.T., Rosati, L.M., Zheng, L., Wolfgang, C.L., Laheru, D.A., Columbo, L.A., Sugar, E.A. & Koong, A.C. Phase 2 multi-institutional trial evaluating gemcitabine and stereotactic body radiotherapy for patients with locally advanced unresectable pancreatic adenocarcinoma. Cancer 121, 1128-1137 (2015).

27. Herman, J.M., Wild, A.T., Wang, H., Tran, P.T., Chang, K.J., Taylor, G.E., Donehower, R.C., Pawlik, T.M., Ziegler, M.A., Cai, H., Savage, D.T., Canto, M.I., Klapman, J., Reid, T., Shah, R.J., Hoffe, S.E., Rosemurgy, A., Wolfgang, C.L. & Laheru, D.A. Randomized phase III multi-institutional study of TNFerade biologic with fluorouracil and radiotherapy for locally advanced pancreatic cancer: final results. J Clin Oncol 31, 886-894 (2013).

28. Hainfeld, J.F., Slatkin, D.N. & Smilowitz, H.M. The use of gold nanoparticles to enhance radiotherapy in mice. Physics in Medicine & Biology 49, N309 (2004).

29. Lee, J., Chatterjee, D.K., Lee, M.H. & Krishnan, S. Gold nanoparticles in breast cancer treatment: Promise and potential pitfalls. Cancer Lett. 347, 46-53 (2014).

30. Leung, M.K.K., Chow, J.C.L., Chithrani, B.D., Lee, M.J.G., Oms, B. & Jaffray, D.A. Irradiation of gold nanoparticles by x-rays: Monte Carlo simulation of dose enhancements and the spatial properties of the secondary electrons production. Med Phys 38, 624-631 (2011).

31. Lechtman, E., Mashouf, S., Chattopadhyay, N., Keller, B.M., Lai, P., Cai, Z., Reilly, R.M. & Pignol, J.P. A Monte Carlo-based model of gold nanoparticle radiosensitization accounting for increased radiobiological effectiveness. Phys Med Biol 58, 3075-3087 (2013).

32. Berbeco, RI., Ngwa, W. & Makrigiorgos, G.M. Localized Dose Enhancement to Tumor Blood Vessel Endothelial Cells Via Megavoltage X-Rays and Targeted Gold Nanoparticles: New Potential for External Beam Radiotherapy. Int J Radiat Oncol 81, 270-276 (2011).

33. Ngwa, W., Makrigiorgos, G.M. & Berbeco, R.I. Applying gold nanoparticles as tumor-vascular disrupting agents during brachytherapy: estimation of endothelial dose enhancement. Phys Med Biol 55, 6533-6548 (2010).

34. Zygmanski, P., Liu, B., Tsiamas, P., Cifter, F., Petersheim, M., Hesser, J. & Sajo, E. Dependence of Monte Carlo microdosimetric computations on the simulation geometry of gold nanoparticles. Phys Med Biol 58, 7961-7977 (2013).

35. Cho, S.H., Jones, B.L. & Krishnan, S. The dosimetric feasibility of gold nanoparticle-aided radiation therapy (GNRT) via brachytherapy using low-energy gamma-/ x-ray sources. Phys Med Biol 54, 4889-4905 (2009).

36. Jones, B.L., Krishnan, S. & Cho, S.H. Estimation of microscopic dose enhancement factor around gold nanoparticles by Monte Carlo calculations. Med Phys 37, 3809-3816 (2010).

37. Mangal, R., Srivastava, S., Narayanan, S. & Archer, L.A. Size-dependent particle dynamics in entangled polymer nanocomposites. Langmuir 32, 596-603 (2016).

38. Tammet, H. Size and mobility of nanometer particles, clusters and ions. Journal of Aerosol Science 26, 459-475 (1995).

39. Poling-Skutvik, R., Krishnamoorti, R. & Conrad, J.C. Size-dependent dynamics of nanoparticles in unentangled polyelectrolyte solutions. ACS Macro Letters 4, 1169-1173 (2015).

40. Lee, I.-C., Ko, J.-W., Park, S.-H., Lim, J.-O., Shin, I.-S., Moon, C., Kim, S.-H., Heo, J.-D. & Kim, J.-C. Comparative toxicity and biodistribution of copper nanoparticles and cupric ions in rats. International journal of nanomedicine 11, 2883 (2016).

41. Jain, S., Butterworth, K., Prise, K., O'Sullivan, J. & Hirst, D. Gold nanoparticles cause radiosensitization in prostate cancer cell lines in hypoxic conditions. International Journal of Radiation Oncology Biology Physics 72, S715-S716 (2008).

42. Su, X.-Y., Liu, P.-D., Wu, H. & Gu, N. Enhancement of radiosensitization by metal-based nanoparticles in cancer radiation therapy. Cancer biology & medicine 11, 86 (2014).

43. Mesbahi, A. A review on gold nanoparticles radiosensitization effect in radiation therapy of cancer. Reports of Practical Oncology & Radiotherapy 15, 176-180 (2010).

44. Richards, K.E., Zeleniak, A.E., Fishel, M.L., Wu, J., Littlepage, L.E. & Hill, R. Cancer-associated fibroblast exosomes regulate survival and proliferation of pancreatic cancer cells. Oncogene 36, 1770 (2017).

45. Shan, T., Chen, S., Chen, X., Lin, W.R., Li, W., Ma, J., Wu, T., Cui, X., Ji, H. & Li, Y. Cancer-associated fibroblasts enhance pancreatic cancer cell invasion by remodeling the metabolic conversion mechanism. Oncology reports 37, 1971-1979 (2017).

46. Xie, J., Zheng, Y. & Ying, J.Y. Protein-directed synthesis of highly fluorescent gold nanoclusters. Journal of the American Chemical Society 131, 888-889 (2009).

47. Rehman, F.U., Jiang, H., Selkec, M. & Wang, X. Mammalian cells: a unique scaffold for in situ biosynthesis of metallic nanomaterials and biomedical applications. J. of Material Chemistry B 6, 6501-6514 (2018).

48. Lodish, H., Berk, A., Zipursky, S.L., Matsudaira, P., Baltimore, D. & Darnell, J. Purification of cells and their parts. in Molecular Cell Biology. 4th edition (WH Freeman, 200).

49. Wolfe, T., Chatterjee, D., Lee, J., Grant, J.D., Bhattarai, S., Tailor, R., Goodrich, G., Nicolucci, P. & Krishnan, S. Targeted gold nanoparticles enhance sensitization of prostate tumors to megavoltage radiation therapy in vivo. Nanomedicine 11, 1277-1283 (2015).

50. Bhattarai, S.R., Derry, P.J., Aziz, K., Singh, P.K., Khoo, A.M., Chadha, A.S., Liopo, A., Zubarev, E.R. & Krishnan, S. Gold nanotriangles: scale up and X-ray radiosensitization effects in mice. Nanoscale 9, 5085-5093 (2017).

51. Kim, M.P., Evans, D.B., Wang, H., Abbruzzese, J.L., Fleming, J.B. & Gallick, G.E. Generation of orthotopic and heterotopic human pancreatic cancer xenografts in immunodeficient mice. Nat Protoc 4, 1670-1680 (2009).

52. Thomas, R.M., Truty, M.J., Kim, M.P., Kang, Y., Zhang, R., Chatterjee, D., Katz, M.H. & Fleming, J.B. The growth of patient-derived tumorgrafts in mice predicts a clinical recurrence after surgical resection of pancreatic ductal adenocarcinoma. Ann Surg Oncol 22, 1884-1892 (2015).

53. Hainfeld J F, Dilmanian FA, Slatkin D N, Smilowitz H M. Radiotherapy enhancement with gold nanoparticles. J Pharm Pharmacal 2008; 60:977-85.

54. Joh D Y, Sun L, Stangl M, AI Zaki A, Murry S, Santoiemma P, Davis J J, Baumann B C, AlonsoBasanta M, Bhang D, Kao G D, Tsourkas A, Dorsey J F. Selective targeting of brain tumors with gold nanoparticle-induced radio sensitization. PLoS One. 2013 Apr. 30; 8(4): e62425.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A method, comprising:
administering, to a patient suffering from a cancer, a composition comprising a compound containing a gold atom; and
administering, to a portion of the patient's body in which the cancer is present, radiation.

2. The method of claim 1, wherein the cancer is characterized by a desmoplastic stroma.

3. The method of claim 1, wherein the cancer is selected from the group consisting of pancreatic cancer, head-and-neck cancer, anaplastic thyroid cancer, brain cancer, liver cancer, and breast cancer.

4. The method of claim 3, wherein the cancer is pancreatic cancer.

5. The method of claim 3, wherein the cancer is head-and-neck cancer.

6. The method of claim 3, wherein the cancer is anaplastic thyroid cancer.

7. The method of claim 1, wherein the compound containing a gold atom is selected from the group consisting of triethylphosphine(2,3,4,6-tetra-O-acetyl-β-1-d-thiopyranosato-S)gold(I), aurothioglucose salts, auranofin salts, aurothiomalate salts, chloroaurate salts, buffered chloroauric acid, and mixtures thereof

8. The method of claim 1, wherein the administering the composition comprises injection of the composition in proximity to malignant cells of the cancer.

9. The method of claim 1, wherein the administering the composition comprises administering to the patient an amount of gold from 0.0001 mg/g tumor cells to 10 mg/g tumor cells.

10. The method of claim 1, wherein the administering the radiation comprises administering X-rays or protons.

11. The method of claim 1, wherein the administering the radiation is performed from 0 seconds to 14 days after the administering the composition.

12. The method of claim 1, further comprising:
determining, after the administering the composition, whether an amount of gold nanoclusters sufficient for radiation dose enhancement have formed in the nuclei of one or more malignant cells of the cancer.

13. The method of claim 1, further comprising:
administering, to the patient, a cancer treatment modality other than the radiation.

14. The method of claim 13, wherein the cancer treatment modality other than the radiation is selected from the group consisting of surgical resection, chemotherapy, immunotherapy, checkpoint inhibitor therapy, oncolytic virus therapy, thermal therapy and two or more thereof

15. The method of claim 1, wherein the patient is a human being.

16. A kit, comprising:
a composition comprising a compound containing a gold atom; and
instructions for use of the composition in a method comprising administering, to a patient suffering from a cancer, the composition; and administering, to a portion of the patient's body in which the cancer is present, radiation.

17. The kit of claim 17, wherein the compound containing a gold atom is selected from the group consisting of triethylphosphine(2,3,4,6-tetra-O-acetyl-β-1-d-thiopyranosato-S)gold(I), aurothioglucose salts, auranofin salts, aurothiomalate salts, chloroaurate salts, buffered chloroauric acid, and mixtures thereof

18. The kit of claim 17, wherein the instructions comprise instructions to administer the composition by injection of the composition in proximity to malignant cells of the cancer.

19. The kit of claim 17, wherein the instructions comprise instructions to administer the radiation by administering X-rays.

20. The kit of claim 17, wherein the instructions further comprise instructions to administer, to the patient, a cancer treatment modality other than the radiation.
